SEQUENCE LISTING (27 sequences)

SEQ ID NO: 1 - protein, 358 aa, Mouse
MTAMLTLETMASEEEYGPRNCVVCGDRATGYHFHALTCEGCKGFFRRTVSKTIGPICPFAGRCEVSKAQRRHCPACRLQKCLNVGMRKDMILSAEALALRRARQAQRRAEKASLQLNQQQKELVQILLGAHTRHVGPLFDQFVQFKPPAYLFMHHRPFQPRGPVLPLLTHFADINTFMVQQIIKFTKDLPLFRSLTMEDQISLLKGAAVEILHISLNTTFCLQTENFFCGPLCYKMEDAVHAGFQYEFLESILHFHKNLKGLHLQEPEYVLMAATALFSPDRPGVTQREEIDQLQEEMALILNNHIMEQQSRLQSRFLYAKLMGLLADLRSINNAYSYELQRLEELSAMTPLLGEICS

SEQ ID NO: 2 - protein, 348 aa, Human
MASREDELRNCVVCGDQATGYHFNALTCEGCKGFFRRTVSKSIGPTCPFAGSCEVSKTQRRHCPACRLQKCLDAGMRKDMILSAEALALRRAKQAQRRAQQTPVQLSKEQEELIRTLLGAHTRHMGTMFEQFVQFRPPAHLFIHHQPLPTLAPVLPLVTHFADINTFMVLQVIKFTKDLPVFRSLPIEDQISLLKGAAVEICHIVLNTTFCLQTQNFLCGPLRYTIEDGARVGFQVEFLELLFHFHGTLRKLQLQEPEYVLLAAMALFSPDRPGVTQRDEIDQLQEEMALTLQSYIKGQQRRPRDRFLYAKLLGLLAELRSINEAYGYQIQHIQGLSAMMPLLQEICS

SEQ ID NO: 3 - DNA, 42 bp, Artificial Sequence
  Primer
gcgcgcgggccctggcatacattaacacaaacacatacatat

SEQ ID NO: 4 - DNA, 42 bp, Artificial Sequence
  Primer
gcgcgctctagaaggacccagactctggacccagggcaaaga

SEQ ID NO: 5 - DNA, 42 bp, Artificial Sequence
  Primer
gcgcgcgtcgacaggtgaagtgcttctccccaacagaaacaa

SEQ ID NO: 6 - DNA, 42 bp, Artificial Sequence
  Primer
gcgcgcgcggccgctgtcctgggagcagcctctgcagccgct

SEQ ID NO: 7 - DNA, 25 bp, Artificial Sequence
  Primer
ggacaacctcagcccacagtgatgc

SEQ ID NO: 8 - DNA, 25 bp, Artificial Sequence
  Primer
tcctttggttaccacctgactctgc

SEQ ID NO: 9 - DNA, 29 bp, Artificial Sequence
  Primer
ccgcctctagaagtcaacattggttagac

SEQ ID NO: 10 - DNA, 29 bp, Artificial Sequence
  Primer
ccgccggatcccacactaagcctcataat

SEQ ID NO: 11 - DNA, 1822 bp, Mouse
accaggaccatggagcccagtgtgctgctcctccttgctctccttgtgggcttcttgctactcttagccaggggacacccaaagtcccgtggcaacttcccaccaggaccccgtcccctgcccctcttggggaacctcttgcagatggacagaggaggcctcctcaagtctttaattcagcttcgagaaaaatatggcgatgtgttcacagtgcacctgggaccaaggcctgtggttatgctgtgtggaacagacaccataagggaggctctggtgggccaagccgaggctttctctggccgggggacagttgctgtcgttgagccaaccttcaaggaatatggtgtgatctttgccaatggggaacgttggaagacccttcgtagattctctctggccaccatgagagactttgggatgggaaagaggagtgtggaggagcggattcaggaggaagcccaatgtttagtggaggaactgcggaaatcccagggagcccccctggaccccacgttcctcttccagtgcatcacggccaatgttatctgctccattgtgtttggagagcgctttgagtacacagaccgtcagttcttgcgcctgctggagctgttctatcagaccttttcactcataagctcattctccagccagatgtttgagctcttctctggcttcctgaagtactttcctggtgcccacagacaaatctccaaaaacctgcaggaactcctcgactacattggccatagtgtggagaggcacaaggccaccttggaccccagtgttccacgagacttcattgatatttaccttctgcgcatggagaaggagaagtccaaccagaacgcagagttccatcaccagaacctcatgatgtctgtgctctctctcttctttgtcggcaccgagaccagcagcaccacgctccactatggcttcctgctcatgctcaagtacccccatgttacagagaaagtccaaaaggagattgatcaggtgatcggctcacaccggctaccaacccttgatgaccgcaccaaaatgccatactcagatgcagtcatccacgagattcagagattttcagatcttatacctattggagtgccacacagagtcaccaaagataccctgttccgagggtacctgctccccaagaacactgaggtgtaccccatcctgagttcagctctacatgatccacagtactttgaacaaccagacagtttcaatcctgaccagttcctggatgccaatggggcactgaagaaaagtgaagcttttctgcccttctcaacaggacaaatttttgatcaaaagtctgtgggaaagcgcatttgtcttggtgaaagcattgcccgcagcgaattgttccttttcttcacgtccatcctccagaacttctctgtggcaagccatgttgctcctaaggacattgacctcactcccaaggagagtggtattggaaaaatacctccaacgtaccagatctgcttcttggcccgctgattgggctgaggcagacaggggtcaccagtaatgttgagaatgactctgtctttgagcctctgagacagctggtggaaatcagtactcctattgcatgtctccaaatctccagggctccaaggcatgttcttcttccctgtgaatggcactggagaaatcaatcaactgtctttcttgacatgtgaaaagagacttctggagtccacatctcatgttgagtcacttcccttttcctcccaatagcccaagtgtccacttatcagctccgcatgatctgggatctgtgctaatggactctgtataaggtctg

SEQ ID NO: 12 - DNA, 42547 bp, Human
gatccaggggtctgccgtagatcttagccatggactgcacctggggctccgtttgaagaactatttgtagttttacagcttcaattctggaagagacaaacttaacaaggaggttaaagacacagggattgaaatgtctggcctgaagtgcaggggattatttctttggcacacttcacaggccctgactacctgcttgatagttttgaaaaggcctggtccagcaaataatgatttggccattggatgggtgctatcaacgcctaaatgaaaggtttggtgaagggttttaagtaatttccattggttagctgcaggcaaaagtatttttccttctttggtggctagacatcctgaggggagggaactatgtccttgtgaggtaccccattctatttcttctgctgagcactggggcttggtttcctggaggggattaccccatactagggttccttctataagcatttctaatggagggtcccgccttgtggctctttggcttcaatatctgcttggtggttcccttctatttccctttcctttcctttctgatgaccccagcagtgtaagatggccacctctttaggtttccgtacagcccataataactcataatggcttcctgatgtttaataggtgttccctcagaagttaggaattccatttctcaccatatttttgtgtaggcatggaggagaaggtaagcataattagagtgtttatatatatttacccttttcccttctcctaattctagtgtatcatggcccctgcttttgctaggatgtctctccctaacaaaggagtggggctttcaggcataattagaaaggcatgtgaaaagagtaaagtcccccagttacaacttagtggctgggcgaagtaactagtgactgcttgtcctaggacccctcggatagtaacagatctggaaatccagctagtcctgtctctcaaaactacaatgataaacaataagagaaaaagaaaggaagaaaggatatacatacataaagaagccaactaataatatgacaggattaagctctcacatatcaataataaccttgaattaaatggattaaactttccacttaaaagaaagagactggctgaatggatttaaaaagcatgacccagctatatgctgcctacaagaaacacatctcaccaggaaagacatatatagtgaaagtaaagaaatgagaaaagatattccatgcaaataaaaaccaaaagtgagcagcaatacctatgtttataaaggaaacagactttaagtcaaaaactgtaaaaagagacaaagaaggtcaatacataatgataaaaggatcaatcgagcaaaggatccatgaggaaattcctgcctaataaattttggtcagaccggttgtctgctctcaaaccttgtctcctgataagatgttatcaatgacaatgcttgcccgaaacttcattgcaattttaatttcaccccggtcctgtggtcctgtgatctcgccctgcctccatttgccttgttatatcttattaccttgtgaagcatgtgatctctgtgacctacaccctattcgtacactccctccccttttgaaaatcactaataaaaactttctggttttacggctcagggggcatcatggaacctgccgacatgtgatgtctcccccggacccccagctttaaaatttctctcttttgtgctctgtccctttatttctcaggctggccgacacttagggagaacagaaaagaacctacgtggaatattgggggtgaattttgcccgatatctggctgaatttcccctgataatgccactctctatgtccatgtgtacacattgtttagcacccacttatgaatgagaacatgtgatattcactttctgtgcctggcttgtttcacttaagataatcccctccagttgtatccatgttgctataaaagacattattttattcctttttatggctaaatagtattcaatggtgtatatataccacattttatttaaccattcatctgttgattccctatttttgctattgtaaatagcatttggaccacatttcaagtacttaattagtggccacatgcagcaagtgactatcacattggacaatgtagccccaacccactgtatgaccttgggtaagacttgcaaactgtcattgcttcaatatctccatctataaaatggggatggcaacaatacctcactaagagtgtaaagactgagttactgtgtgtaaagcacttcacgcctccccatcggtgcttcaccctggggctgcaatgagcacccaatcttagtgtcagatgacacagcacagcaagaccgaggcccttggttcaggaaagtccatgctgccacctcttcagggtcaggaaagtacagtttccacctcttacaaataggactgtttgtctgctcctcctgggtcaaagtaacttcgggttcaggtcctggatccagcaaagggtttgcttaacattgcaagaaagatgttgcctcatggtcaaaagtcaggcgtaggatgagacaggcagacacgcacacattcacacccacgttttgcaaagatggactgaccctgtcagaggatgtgtgggtgaaggtgcacagtgaggatagagacatatgggagtccagtagacatcaatcaaactggactcagtttgcacacacctggagctcaagagtctccagggggaaaacagagacacaaagtcagacagagagagagccagagaaatttcctgcaccgtgaagatagtcagaggcagggaagaaactccttagcactagttagagtgatcagaaaccaagaggacctgatcgctgtacctgccaggtctcagtttctgtctccttccaactgaccacctcttcctctgagactcaccagttctgcatctcttgctcctccttctgtttctccgaccacttccacctgtggctgtcacagaagggcggatgaaggaggggacactggagatagactcagcatctgcaggcttccaaagagaggggctaggagatccaccaacacaccagcacaaatacaccagcacacacagatacacacaattggttcatgtattgctaggttacagtttgctatgctacaaaggcagtaggccaaatttgattgaattgaataattccttattttcatcagcttctcctttttttttttttttttttttttttgagatggagtattgctgtgtcacccaggctggagtgcagtggtgtaatcttggctcactgcagcctccacctcccaggttcaagtgattctcttgcctcagcctcccgagtagctgggattaaaagtacccaccatcacgcccggttaatttttgtgtttttagtacagatggggttttgccatgtgggccaggatggtctcgaactcttgacctcaattgatctgcccccctcagcttcccaacgtgctgggattacaggtgtgagccaccgcacccagccagcctctcagttttgaacatgcactaccaccacctccacaacacacaaatgtaaatgcactttcgtatataaaactgtataaatacaaggaagctcatacacatgcaaggatacacacataagcacccccagattcaaccacagaaatatacgccagtacatttgcataaattcaaacacccctttacatgtaaaaatcatataagcacatacagggatgcaagcaggcatggacaaatgcatgcaagcacagacaaacagacaaagctaagtaaaaaagtgcaagctcacctatgcttacaaaaatagacatacatatacccacaaacccacacacccacacattcacttgctcacctggactttgatatctctaccactgtatccctgccaatatctacagagtgggtaaagggataggcatcaggtcactgggttgcccaagcaggaagtctgggttccctaacaactttttctaagctaatgctcctggatgatgatgaaaaaggaggtggggaatggatgaaattttataacagggtgcagaggcagggtcaggataaaaggcccagttggaggctgcagcagggtgcagggcagtcagaccaggaccatggaactcagcgtcctcctcttccttgcactcctcacaggactcttgctactcctggttcagcgccaccctaacacccatgaccgcctcccaccagggccccgccctctgccccttttgggaaaccttctgcagatggatagaagaggcctactcaaatcctttctgagggtaagacacagacgaatggggtctgagggtgagctgcttcttgccttggtacttggggaagcttcaccaaacagaatgaggcagacttccagagtcaggggtggcacgggcatggttggtgagtacggagcatggtgaagcatgatgggtggtattattaggagaaaagcatcaaattaaatttagcagagtttatttgagcaaagaagtgactcatgagttggacagctccctaaaccaggaaagacaccacacggcagtatggtcaagtggtatttacaggcagaaaaaggaggtgacatacagaaacagcctgattggccacagatcacagcttgccttacttggtcacaatctgagcagtttgcagcctgtgtggactgaaagcccagctgctctgattagccaacacttggctacttgtcacaagaatatattcatttgggccaggtgcagtggctcatgcctgtaatcccagtgcttttggaggccgaggtggtggatcacctgaggtcaggagttcgagaccagcctggccaacatggtgaaaccttgtctctactaaaaatacaaatattagctgggcatagtgatgcgtgcctgtaatctcagctacccaggaggctgaggaaggagaatcacttgaatccaggaggcagaggttgcagtgagccaaaatcttaccactgcactccatcctgggtgacagagtgagactccttctcaaaaaaaaaaaaaaaaaaaagaatatactcccaagttaggttgcagttcactctacagagagagctttaggtcaaatttaatttaattaaacaattctccccttttggtcagcctcaaaattttgagattgaccaaaaccttgggcatcaacattacttctgtcaccatcataatggacttgtctgctctcagtatggaattcacaatggacaatgtcaacgtagttgagtgattctttaccttttcttcatgtttttgttgttcccactgtaatgagcccactggatgtacaaagaatggctgcatatgagcatttaagactctttttttttctgagacagggcctcactctgtcagccaggctgaagtgctgtggcatgatcacgtctcactgcagccttgacctcccaaggctcaagtgatcctcctgcctcagccccccaagtagctggaactacaggtgcatgccaccacgcccagctaatttttgtattttttgtagagacagggttttgccatgttgcccagactggtcttaaactcctgggctcaagcaatccacctgcctcggcctcccaaagtgctaggattacatgtgtgagccaccgcacccggccaagactcttgagaaaatacaacacatcagggagactgttatgatggctctcaggagggtaatacgaagaaaatgaagtcactgggcctgtaataaactttgaggaatgtggacttgggggtatagataaggtccactgtccacagagagaagaaaggctgttaatagtctcttttaacttgagtgtgtccatgaaccaaactgatcaaaatcgaataattcgaagttcagacaataaagatagttcaatagtattagagtccaattggtcatagattttgttcagggcatgatggtaattaaggaccagagcttgctataaaataacttgatttatagagacattcatttgtagttggcctggtaacatatagtatcctggagacccactagaagaaacattaagagtagaaaagtttgggatagccaggcttgctgtgttagtccattctcacactgttataaagacatacctgagactgggtaatttataaacaaaagggatgtaactgactcacagttccacatggctggggaggccccaggaaaatacaattcatggcaaaaggtgaatgagaagcaggaaacttacaatcatgatggaaggtgaaggagaagcaagtaccttcttcacaaggtggcaggaaaaagagagagagccaagggggaagagcctcttataaaaccatcagatcttgtgagaactcactcactatcacaagaacagcatgggggaaaccgcccccaggatccagttacctcctactaggtccttccctccacacctggggattacaattcaagatgagatttgggtgaggacacagagccaaaccatatcacttaccatcaccattcaggatgcttgcaaaccaactgctagctgcacctgtaaacacatatctgtttctttcccctgagaaatgtccttagtgtatttgtggcagtgtctagagaaacagcagtgtcagccgcattttaaattaagttatctgcactagtgaattcactggaaagataagagcaatatttggttttcttcagcaccatacacaagcctccaagatgggcatagaggagatctaaaattgcgtgatgttccattaagcgtttttgttgccacaaatgttctcatctcagtttggagagtggcttctacccatctgaactccttggaggttcaattagctgcaaaattcaagatgtcccttaatgtataacttagcctcagattccatacaactgtcacccaaataccaccaagaatgagcacccaggaacccaactggaaccttttctgaacagaaaccaacttatcttcgtcgattttgaggttgatagtaatttcagttattgactgttttggcttttaactatgggaggtattaggaaactctcagggaaacaatttggaaagcagcagtgagctaggccaaatagcaagttctggacctgtgaggagaaagaacagagtaagcaaacctcaagatactcaaggtaggcactcgtggtgttggaaaagagggtcacctactggcattagagcagagatcagttagatttgtttacccataagtctgcatagctcctgaacaaggtgggaaacttacttttttgtggtctttttctagcatgctgcgaaggtgcataaccacatttagttggaaagagactttactgtatttacttatttatttgtttttaatagagttggggtcgtgctatgttgaactcttggcctcaagcaatcctcccatctcaatctcccaaagtgctgggattacaagcatgagccaccatgcctggccactttacatatttaatccagtaacattacacacgcaattgcccacacccccataggtagtccccaggtcttgcatacgggatgcctggaagcaaaatatgccttttgcagccattattcagatacatttcctatatttagtagtgattatgttattagctagttaatagtatgttattacgtactgttattatattaactaactaataacataatcactactaaatatttccagtgagtgcaaaaaagcaagtggcaatgatgtctagaatatcaagatatagctttccactcctcctttggggtttctgggtgattctcattgggaacatgaagaggcattggcaccagtgaaattatttcctgattttggggcgttggttcagaaactcaacaactccttgttttttttgtttgtttgcttgttttgctagagtataagcctttgctaaagccattcacagattatagtcctatggattttcttgtaaggaaagggaaagggttaggacagcaagaaatggggaagaaaggataaaagataatgctttcatgatggaagagaaatcttgatccacaatcttggaaaagctgtccgcatataagatgccaactgcttctggggaaaaacttccctggtcagctttgccttaaggtctccaacagacatacagttctaggagtctagaagggtcctttccaatggagagatgtggatccaagatccgagaccctgacattttgctacagagaagaacttggcattgtccttcccaatggagtacaaggaacagtcttagaagaacttggtacggtctcttccaatggagttcaaggacagtttgtctggtgtcatttccaaagggcccaacctctaaattctagatcatgaaaggtctggttgtcatcaaccgatgtgtcatcaatgactcattttacctggtgaaaacatgctttggcataaagtattatagccttgcattattgagtcatatcagagtttataagagtgggagatacatgagattctattattaggggcataggccctctattactattttacaagagatctatctatgtctttccagtaggagtggatctgattgccatcaatcaataatacctgagaccaagggactccaatcaattcagcatgctttgcctaatgatatttgtttgtaatactgttgcgggacaatcaaagactggagagaccaaaaaaggttcaggagagtttattaaattaaggtgatcaccggttcagccagacatacatccagaaagtctgagccccgaacaaaggcttttcctacttttaaacatattaaggtgggaactacatgaggcaggaagccagtttcagaagtgagaaacaaagcagttaaataacatttcttacatcttgagaaagacatgtcttgcaacctaaccttatcggtccggtgaccctgcagctgtgcaggaactcactgggcctgtaataaactttgagggatgtggagttggggagtataggtaaggtccactgtccacagagagaagacaggctgttaatattctcttttaacttgaatgtaaggtgtggtcatactttgcagcaaccttaagaggattttaaaatttatattactactactattaggttatagttgatttcattaattccttcttcaataccttatttaactgttttaccacttgtctagtgaaacaagtacctctgtcactggagagttctccaggaatgcccagtaaggaaatacattttctaataaccttttatctacggttatggcattgattgatctttgtgcatagaaatgctttaatacaaccagaaaacatgcaatgaagctggcagttgaattaactccagcttcaagtgttcaaatgatctaccaagtgccagaaatatatcacctgaggtttttgttgtcttactagaattatggatttgataaaccaaatattagttataaaccatttagtaatcttagaatagtcacctcatcaatattttttcactgtttggatcattttctctcttctatgatgagtcatggaatacggagcttttagtaatggaaattttaagaactcaggaaggaccaggcggccatctagggtctccatgagtgcatgcttcacattggaattacagactctaaagtacaaattttaatacaatgagttgcaatttatgcttctctaattcaggtacataacactggtttattaaataggttatcataggtaatttgatggtgccattgcactccagcctgggtaacagattgagaccctgtctctgaattggaactgcacgggggcactgtccttggaggggtgaatgggcatgaagaggtgtctgggtatgagccacaggtatagaatttcactcttctctgccatcctctgttacatcctgggtacctgcctgccactgaaagaatgaggtaaaagaggtggtggcacgaatcaaatagatcttgctgtgccaatgagagagagcagactagcccatgtcagtgccaggagagtggaggagagagggagagcaggagaggagtgtgggtagggagtgctcatcaacagtacacatagtgccctataccggtaactgccactggctcagtatttacctgggttatcactgcttaccatgcctgattttatgattaattatctacttatcattactaatccatcaacccactttccaatgggagaattagaacactgacaataccttccagctcctcttccccttccccctcccatgttgaccatactcctgaatcttaggctcgttatccttttactattaatagagttatttttttaatttctgatcaatggtctttttaatgataccaagtacagagtatatatgccaatactactatgaatttttaaattatttgctcagacagaatacatggacatacaaatgatgaatgtgataattgtcatacatatatatttatcttagacacttagtcaaaacatgtggtctttggttatcagttagacactgtcactttacctggaagatacaaggtaattggtcacagactctcaaattatgaaacatttagatttttcaggggaatgacatggagggagccaaggagtcttatgattagataagatgttgtttgggtggctcacgacaccactgggcaacactcaaagaggtggtggcttatacctgtaatcccaacactttgggaggctgaggtgggaggatcgcttgaagccaggagttagaaaccagcctgggcaaccaaacaagacctggctctacaaaaaagttttaaaaatttagccaggcatggtggcatgtgcctgtagtcccagctacttgggagactgaggcaggaggatgacttgagccttgtagtttgaggctgcagtgagctatgatcacgtcactgccctccagcctgggcacagagcaagaccctgtctcttaaaaaaaaatcatctgcaatgtgaggagtgataacatttaggaacgtgtgtataggtttaaatgctggtcaaagacatcctacacaattcgctgaaccttctctctaagggttttttcccaagctctgcagacgctatctgggcacaaatcatgcctctgttaacagaatttgctgttccttctagctcttggtatcccactgcccgctttctttatgaaagctggtatggtcattgaatatcccaatcctttacaaatttggaaacaagcaaaactgtcaatgaaatttgtatttgcctaaaatgagttttctttctttcttttttattattattatactttaagttttagggtacatgtgcacattgtgcaggttagttacatacgtatacatgtgccatgctggtgcgctgcacccactaactcgtcatctagcattaggtatatctcccaatgctatccctcccccctccccccaccccacaacagtccccagagtgtgatgtttcccttcctgtgtccatgtgatctcattgttcaattcccacctatgagtgagaatatgtggtgtttggtttttttgttcttgcgatagtttactgagaatgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacatgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcttctccagcacctgttgtttcctgactttttaatgattgccattctaactggtgtgagatggtatctcattgtggttttgatttgcatttctctgatggccattgatgttgagcattttttcatgtgttttttggctgcataaatgtcttcttttgaaaagtgtctgttcatgtctttcgcccactttttgatggggttgtttttttcttgtaaatttgtttgagttcattgtagattctggatattagccctttgtcagatgagtaggttgcgaaaattttctcccattttgtaggttgcctgttcactctgatggtagtttcttttgctgtgcagaagctctttagtttaattagatcccatttgtcaattttgtcttttgttgccattggttttggttttttagacatgaagtccttgcccatgcctatgtcctgaatggtaatgcctaggttttcttctagggtttttatggttttaggtctaacgtttaagtctttaatccatcttgaattgattttgtatactaaaatgagttttcaaaaggatctttgtggctaccttattagttcatagaaagtggaggcttgtctggaatgatattaaagaatttttttcattttaattgttttagagacagggtctcagtctgttacccaggctggagtacaatggcacagtcatagctcactgcagccttgaactcctgtgctcaggagattctcccaccttagcctccagagtagctgggactaaaagtgtgagccaccatgccccactatttatttttgtagagatgtgtttgggggacggtctcactatgttgcctaggctggtctcgaactcctggactcaagcaatcctcctgcctcaacctcccaaagcattgggataagttttgcatagacatgtttgaccctctcccttcctttattgcagcaaagatttccattttctcacagggaggacttggggcaaattgtttttgtcgttgttattgttatttgaggatgtgggtgggtcatcttggttaatgtcagtgcaagaacggtgtctgctcctcattattggggttccttagctattcaggcaggcagcagctcccttgagaagacttcctgacccccaggtccccatcatatgctctcatacaccagctgcccctccttactgagcctatgtccttgatagtgctgcatttacctgtgatcctgtggctgagctctccctcacacttccagacaaggaaggcctgtgaggtatcagcacagtgctgaacatagtacctggtacacaataggcatttagtaaatatgtgaacaggaacaaatgaaggagtcagtgagtgaaagctccaagcctgactcagcggaactggcagtcggccagggcctacaaagtgctgcctggccctcagtagggggtggcagatctggggatccctcttcaccaaatagagttgcatcatacaggtaaaggtcccaagtgcctattgttttcttttcatcattttccatgtgtgacaagagtatcacgcaatcatgtgaatcaatggacttagttttctcactagactaatgtgtctaggaattatctgtgttgtcatggggattttccaggtgtcatttacaactacctgtggacaagatgaggtgctaccctcatcttagaattagggatggtggctgggcatggcagcccatgcctataatctcagcactttgggaggctgaagcaggtggatcaactgaggtcgggagttcgagaccaacctgaccaacatggagaaaccctgtctgtactaaaaaacacaaaactagctgggcatggtggcacatgtctgtaatcccagctactcgggaggctgaggcaggagaattgcttgaacccaggaggcggagtttgccctgagctgagattgcatcattgcactccatcatgggcaacaaaagtgaaactccatctcaaaaaaaacccaaacagaaacaaacaaacaaacaaaattagtgatagtgatgctcagcctggggaaagcatttgtccagtggcacacaactgggaaaaggggaagctgagatccagcaggaggtctgtctccaaagccctcctagactaaagctgcttaacaatttgtggattatgaaattctcatagagtttgattaaagctgtggccccctcttttccaaataggcacatacatgtttgcataaagttgtgaggcttcacacactccctaaaactctttcataaaccttccaaggatcctctaggtattcacgaccatctattatgattgcatctcttgggggtggggtaaagagggagggcatgagcaagtgtgcatcagggctgaggaaggtggcgctgttgcttctccatttcccaataagcttccagattctttttgatgtcagagggatgtgggctcgtgtctcagattcaacccgtatgcattgagtcacagttttctcttccgtcagttaagatcatgacaatgagaatgtgtgcccctaaggtggttgtgaggattaaatgggatattgcatacagctagtatataataagtgctcattaaatggcaactaccttgatccactcattcatttattcacgaatccaataacaattcactgggcactttctatgtaccaggaaatagtctaggaattgatgatgtggcatcttggacaagacatacgaggtcactgcgcttatggacattccattggcagagacagataagcaaaaaataaacagataaggaaatgttaggtggagaagagctacaattaaactaaagcagggggatatttcagacagtgatgagaactacgttagattgagtggttaggggaagactgtcttgtttttttttctgagcagagacaatgaaagattcaggtggaaggcactagaaggaggcaggggtggctacaagaatcctgaaacaggaaaaatgtcagagaacaacatggaggaatgagtgggaggagaagtcagagcggtaatagggccacgtcatgtaggaccatgtgaaccctcagtggagacttcagatcttattctcagcaaagtgggaatattgtaggccttggtgagcacgtggatattaattattttacattttagtggggttaccctggcttgtggggcagtgaaacaattgttaagtggagtaagcatgtcatccagaagtccagtgaaggggctttgcagagatcaatgcagggcatttgtaagtaaacttaacaaattttatgacttttatgtcaagtttttattgaacttgaacatcactgtaaaaagcgcacagctcaatgaattttcacaaactgaacatactcatgtaattgacatgcaggccaagtagcaagaatgccccgaaaccctgcaccctgttcttttctattcactatccacctaccatcttgatcatgggttgattttatctgtgtatgaacttcttttttgtttttgtttgtttgttcctgagattgaattttgctctgtcgcccaggctggagtgcagtggcactatctcgcctcactgcaacctccgcctccccgggctcaagcaattctctcgtctcagcctcccgagtagctgggactacaggtgagagccaccatgcctggctaatttttgtatttttagtagagatgggttttcaccatgttggtcaggctggactcaaactcctgacctcgtgatcagcctgcctcagcctcccaaattgctgggattacagatgtgagtcaccacgcctggccctgtgtttgaacttcatacacataaaattatgatgcattgactcttttgtgcctagttgcttccactcaacattatgcctgtgagtttcagccacgttgtcgcctgtagctgtggtttgttcttttttgttgctgtatactagtctattgtgagaaccctcactgattgtctatatctatattgatgagcattttattaagaaagcttctgtgaatattttgttcattttttggtgaacacacatacacatttttgctgggcatgtacctgggagtgaagtggctggtttcctgggtatccatttgttcagcttcaggaaatactgcccagcagttccccaagtggctgcacagtaatcccaccttatccactggagatatcttctgagacccccagtggatgcctgaaaccccacataatactgagcactatgcatactctttttttttcctatacaatcacattatatagggagggtggtatacacagtgcagacatggtggacaaaggatgattcgtgtccggggtgggacagagtggatggtgagagatatcatcatcctactcagaatgatgcacaacttaaaacttacgaattctttatttctggaattttccatttaatattttcagactgcgattagctgcaggtaactgaaactgcaaaaagcaaaaccacagatcataagaagtatgcggtgggggtgatattcatcgtattttatcaaccatctttactgtttagggcacaagccaatcagagcagaccctggctgggccacccattaacctaagcttgtccaacctgccttattttgttgttgttctgttttgttttgttttagctttttagcagcctgaagccatggttttcagtttctgtctccagtgatacacagaaaggaaggatgaggaaggggctttactggcccaaccagaaacggaaactaagaacccatgactgtattctctcccttggacagcgttaaccattaacccttaattgctgggtcccagcaggggaaagggcagcctggggaggcggatgttggggaggggctaattaccaatctggtatgtaagtattttgatagttttacaaatgaggtgtatgctgactaacagccacccctggtgtggatgtgattggcagttccgagagaaatatggggacgtcttcacggtacacctgggaccgaggcccgtggtcatgctgtgtggagtagaggccatacgggaggcccttgtggacaaggctgaggccttctctggccggggaaaaatcgccatggtcgacccattcttccggggatatggtgagagcctcagaggcactgggagggggcgggtggggggtgcatcagggaagggagtatatgggaggaagaaggactcagagccttcttccaacttcttctacaaccaacccacacctcccctgcaccccaggtgtgatctttgccaatggaaaccgctggaaggtgcttcggcgattctctgtgaccactatgagggacttcgggatgggaaagcggagtgtggaggagcggattcaggaggaggctcagtgtctgatagaggagcttcggaaatccaagggtgagtcctgggggatgaataggaaagaaagacaatgaaacactgagagatgcaggtgcacgggaatagaaagacagagaggtatataagggcacagacagagacagacgaaactggagacaccatcagacagagggatagagacagagagggagagagacaggggaatagagagggatggggatgggcaggagagaaacacagagagccagggaaagagagagatgccaggtgtataatgtccaagagttactcaaagaggctggatgtgatgactctcacctgtaatcccagtactttgggaagctcaggcaggaggattgcttgaggccaagagttggagaacagcctgggcaacataatgagatcctgtctctacacaatatagaaaagaagtgagccacgcatggtggtgtgtgcctgtagtcccagctactcaggaggctaaggtgggactacaggatcacttgagcccaggaggttgaggttgcagtgagctgtgattgtaccactgcactccagcctggacaacagagcaagatcctgtctcaaacaaacaaacaaaccctcaaagacatataatttcatggatcaattgtgtctgtcaaagtcaaaaacggaagttaagtaaaagaaaaaaactacagacatttaacaaataatgaactgtgttttccttgccctgggtgaagtgctgatgagctggcagtgagcagacaggccaggtggggtgttctgcccgggtgcagctggaggggtcatcaaagaatcactaggttatttttgagttctccataacttggtgtctgtgagacatgtaggtgaagggtctctggctagcacctccatctcattcatgccaggtgtttaccatctctcttatcaaaatttctcaagagactctgggatgtaaatgcagaggctgcatggggaggtagagacccaggagctatagggaaacggggacaagaagactgaagagaaggacaggaagaaacagtgacacaggcaggaggaaagagacagatggagggaccaaaacaaaaaaatataggttgggtatggggctcatgcctgtaatcccagcactttgggaggctgaggctggcggatcatttgaggccagcagttcaaaaccagcctggccaacatggtgaaaccccatctgtgctaaaaatacaaaaattagccaggcttggtagcacgtgcctttaatcccagctactcaggaggctgagacaggagaattgattgagcctgggaaatggaggttgccgtgagctaagatcacactactgcactccagtctgcatgatagagtgagactctgtctccaaaaataataataataataataaaataaagaacggcaggggggagacaaatatacacacagagagacagaaagaaacaaaggcaaagagaaattgaggcagagaaaattagagagacagacagacaaagcttaggaaaaggtctgcagaggaatgagagaagacaggcaagtgagaaccagagagaggctgcactaacctgatgttcttgggtccttacagaccactctccctccagctggggccagtgctgagcctggtgtatacaggtatcacttaacaagtacagaataattcccagaagactggagagccctagatgtggaaagaagagattaagggggagtaataggtaggggtggaaagatggttttttatttgttttaaattagagacggggtctcactctgtacccaggctggagtgcagtggcacgatcatagctcactgcagcctcaaactcctgggcccatgtgatcctcccacttcagcccctggactatttcaactgggactacaggcatgtgccaccatgtctagctacatttttttttttttttttttgtagagacagggtctccttatgttgcccaggctggtcttgaattcctgatccttttgaatcaggctcccgaagtgctgggtttagaggtatgggcccctgtgcccacccagggctttttaatttatataagcaattgattgaacacctactctgcccagcccctatccctgggatttaactgtactcactcccagagtcagaggtggggcctgagaggaggtgcagagtgagaaccggctgcatggactctatagctgtgttgcctgggtctaaatcctggcctcagtaatgagtagctgtgcaactttggtcaaattactcagcctctcggtctgcccatctataaactggagctaataatcaaattgcatctgcctcacattgttgtagtgagagttcaatggaattacgcgtgacgtgctggtacataattagctgttacggttattctcatgtttaccattactgagtgatggcagacaatcacacagagataggtgacagcctgatgttccccaggcacttcagtctgtgtccttgacctgctgcttcttcctaggggccctcatggaccccaccttcctcttccagtccattaccgccaacatcatctgctccatcgtctttggaaaacgattccactaccaagatcaagagttcctgaagatgctgaacttgttctaccagactttttcactcatcagctctgtattcggccaggtcagggagacggagagggacagggggtgtgggggtgaggtgaacacccagaacacacgagaaaaggatgacctgtcttgggggctcagaaatgcagcttatccttggaagaaacgcagacatgtgaagaatcagggacatggagacctggagggaggagagacggtgagacagggatagagacactgagagagagaatgaggcgtgatggggaggcagaaatagagtcagagagagactgagagaaggaagatgagcaaaaacaagacaaagaagagcagaaatcaagagattctgagagacagagttgatgagaatgagtgtgaaagagagggagagagagagaacgaataaggctttgggcttcatgtctattctgctcctggatgtcatttctgttttattttttttagacggagtctcgctgtttcattccagctggggtgtagtgttgccatcttggcttactgcaacctccacctcccgggttcaagtgattctcctgcctcagcctcccaagtagctgggactgcaggcatgtgccaccacacctggctaatttttttttttttcttttcgagacagagcctcgctctgttgcctaggctggagtgcagtggcacaatctcagctcactgcaacttccacctccctggttcaagcaattcccctgcctcagcctcctgtagctgggattacaggcgcctgccgctatgccaggctaatgtttttgtatttttagtagagacggggtttcgccatgttggccaggctggtctctaactcctgacctcaagtgatctgcccgcctcagactcctaaagtgctggaattacaggtgtgagccaccatgcccagactgcttctggttcttctgtatccttgcttctcagtctttggtaaagctctccacctaaagaaaatgaaggataaatgacaataaggaacagcatttcttcattttctcccatttctccttctccctctgtgtttttttttttaactttccccagattgtaaaggcagtcttctgctcttttaaaacaaaatactaaaatgtctccttatttattaacctggaaatatgcctattacatattaaatttaagaatatcaagctgcagaacagtatgcatagctgtagtttgttgttgttgttgttttcagacagtatcttgctctgttactcaggctggagtgcagtagtgtgatctcaactccctgccacctccacctcccaggttcaagcaattctcatggctcagcctcccgagcagccgggactataggcgagcgccaccacacccagctaatttttttgtatttttagtagagatggtgtttcaccatgtcggccaggctggtcaacatagctacagctattaagcagggatgtatgttggattcacatgtggggttgtcacagttatggattttcaggaccctactttctgggcggtctgatctggaaagtctgggatggggcccaaggtgagtacttgtaacaagcccgaccagtaattctaatgttctcctcccaccgagaaccacagagaaaagtctggaaggagacccaccagacagttaacaatggttatctctagaaggagagattaagaaggaaatttacatctgactatatatgtttgcatttttgcaattatttgcaataaattaggcattccattcttcatcaaagtaatagaaataacctccaaaatacgtagtcctaacatgtcagcaggcttatcttgtgtaagaatcattttattaatatctgacacagcaagggagatgaggagaggtgggaagagggagagaaaagtatgagaaagacaaataaacaggctgaggtagacaatgggtgacacagaaaggaagtgagacagagactaagagagatagaaaggagagaggcagggagatggggcagaggccaagaaaaagacagaaggatgagggaggaagatgcagaaagaggtaaatgtgagatagatcaaaggagatatagagtcagtgagtgaggggttcagaggcagaggggagtggggaagtggggttcccatggagggattggggcccaggaggcgctctctccctgtgacctgctagctcagccctaggcaaacctcaccaccccttctttcttgcagctgtttgagctcttctctggcttcttgaaatactttcctggggcacacaggcaagtttacaaaaacctgcaggaaatcaatgcttacattggccacagtgtggagaagcaccgtgaaaccctggaccccagcgcccccaaggacctcatcgacacctacctgctccacatggaaaaagtggggtctgggagaggaaaaagggaagggaggggagggagggcaagatggagaggtgagaagagggagggaaaaggggtagggaaggggaagatggggagggaagaagaaagactagggaggggagaatagggaaagggaggagagaacatgaggaaggaaagaaagatgaggtgaaaggagggagaaaatagggaggaggaactgagacagggagagaggggaggtgggaagacagaatgaaagacagagggagagagagagaagactggctgaggaaggaattcggggcaagggacaaaaatacagcaacaagagaaaaaactcacagaggcagaaagagacggggacaaaaagagagaaacacatcaaagagatgtggagagagatagaaacagagttaggaagactaaagagaggctgagagagatgagttagagatacgcggttggatgtgtagaggacagagaaaagcaaactgggccagatagtgtcaaagacctttaggccaacggagggcagccagggagatgggcgtatacacagcaaggctacagcctcccctgaccctccccttccttccctactgtggacgcaggagaaatccaacgcacacagtgaattcagccaccagaacctcaacctcaacacgctctcgctcttctttgctggcactgagaccaccagcaccactctccgctacggcttcctgctcatgctcaaataccctcatgttgcaggtgggccagggacagccagtcaagggggtcttctgacctccttctgagctgcagaaatggggctatgggtaccacctggatgagagaggggatgctggcttcctattctgggagcactgtaggctctgggctagattccaaccaagccaattctgttggtggatgcatggatgcatgaagaatctgtccatgcgttctcccactgttttcttccatcacttaaggattttttgttctaaggtttttgtttgtttgtttgttttttgttttttggtttttttttttttgtcttttttgagacagagtctcgctctgtcacccaggctggagtgcagtggcatgctcttggcttactgcaagcttcacttccagggttcacgccattctcctgcctcagcttcccgagtagctggaactacaggcgcctgccaccacacccggctaattttttgtgtttttagtagagatggggtttaaccatgttagccaggatggtctcgatctcctgacctcatgatgtacaaatttagggggtacatatgcagttttgttacatgcgtaggttttgtaatggtcaagtttgggctgttagggtatttatcacccaaacagtgtacattgtacccattaagtaatttctcatcattcaccccccttctgctccctcactcttctgagtcttcactgtgtatcattcctctctctgtgtccatgtgtacacattttgtagcactcactcatgagtgagaacatgcaatatttgactttctgtgccatcacttaaaaatcgatccatccacttatcatttcatctattcattctttaattcattaattaaagaatgtataattactgattctttcatttatgattcatctaaggacatatactgtcattcatttatttggattacttgatccacaagttgatcctttgaaacagtggtatgttgatggactatttgtcattgattcattggctcattcattcattcattcatgcattcatccatccttctacgaactaggtttcactcttatccttccatgagtcaacctttcaattcacccttaatccatccgtgatttccttcatttatcacaataattcactcatttattcacctctgatctacttatcattcaatccagcctttcatttactcctttatttactcatacttcactaatttaattattcacttgctcttccataaatctagccattcatgtgattattcattaattgggttcattgatttctttgtctatggatcattcattagtgattaattaatcaatccatctattaattgataagtaaatacagacatccatttattggtttgttcatttattcatcaatctttccatccatgaattgatctattgattgattgattgatgtttttatccagttgttcatgaattcatctattgttctattacactgttatatacctgaggaccaagaatagtgtctcaactatattataaacacaataaatattagttcattttctactcatcttagagagggtgttttgagaggtttgtagcctggagttcttaatctgaaattccatgcaaaatcgtggtgtgtgtgtgcatgtgtgtgtgtgtacctgggcatgtgggaagaggatctgtaatattcattagatttcaagcagtgagaattcttgttgcttcctccctcctcccctcaccccatgctgattactttgaggggtatcaaggatctaaccctttactataggtttttcattggtcaatagaaagtagtgtccttgctgaaaggtctctttttaaaaaaatttttttttcttttgagatggagtcttgctctgtcacccagtctagagtgcagtggcatgatctcggctcactgcaacctccacctcctgggttcaagtgattctcctgcctcagcctcctgagtagctaggaatacaggtgtgcaccagcacacccagctaattttttgtatttttaggagagacgggattttgccatgttggccaggctcatcttgaactcctgacctcaaggaatccacccacctcaacctccaaaattgctgggattacaggcatgagccaccatgcctggcctgaaatgcctctttaaaatgagattcattggtcttcttttctgtacagagagagtctacagggagattgaacaggtgattggcccacatcgccctccagagcttcatgaccgagccaaaatgccatacacagaggcagtcatctatgagattcagagattttccgaccttctccccatgggtgtgccccacattgtcacccaacacaccagcttccgagggtacatcatccccaaggtaagaccggctggaaccccatagccctcctgtttgggcatcctggattctcttaatccccgaactcaaccttttgttagctccttaattgagtcccgttgtttttgttttttgtatttcttttttgtggagtgtgtggagggttggagggaatggcaatatcttttgatcttgtgatcctccctcaggacacagaagtatttctcatcctgagcactgctctccatgacccacactactttgaaaaaccagacgccttcaatcctgaccactttctggatgccaatggggcactgaaaaagactgaagcttttatccccttctccttaggtaagctggacccacaatttctttcccagacaccagagggcaggtactatccccaacttgagaaaaacaacgagagatactgattatttgagcacttaatatattctgattgcttcacctgccttatcccattccatcttcactacaaccctataaggaggcttgagaaagaagattacattcccaaaggcacatcttggcaagcaggaccttgggcaagtattttaacatctctaaacctcagtgagttcattttcttaaaaagaaaaaatctgttgggcaccactgtaagcccagtgctgtactgggggctgaagataatgcatcaaacaagtcacacagagacagggttcctgccccaggaaatttaaagtccagcagggaagatgggcattcatcaaataataataaaataatcatctcatgaaatgaatgaatgcctgcaacatgcttagaactgcctggcacaaaggacatgctcacaagggcaattattattataattagacataattgtgataagtgctctaaagggagctttgggagcacaaaataggaaatagtagctaatcttgtggtgggtccgtaaggaaaagcttaccagaggaactggcttccaagctaacatgttcatgggtgagcatcaatcaaccattgcaaagtgtgttccaggcaaaggaaacagcaaggacaaaggccaaaggtagaaacgtgatatggcaccattgagaacctataggaagtccagtgagccttgggtgtagattgcagaaggaaataagacaaagggctcatctgggcaggaccttgaaggttgcaggaggagtttggatttatggtctagcactgggaaggtggaaaaggtcttgacgtgctctgacttgtcccagttctcatcctctactctttggctggttaaaagaaaaactttagacaaattaaacttagcagagtctatctgaacaaagaaacaattcatgaattgggcagcacaaggaaccagtaaaggttcagagagctccatccagcaatgtggtcaggcactatttatccacagggaaaggaactgaggtactgaaacagcctgattggttacagctctgtgtttgccttatctgagcatgtctgggcagcttgtagcctgtgactggctgaagcttggctgccctgattgtccaaggttacttgttacaagaatatactctcaagtttgtttacatgttgagttacattacaatttgttatgtagggaggctgctttaggccaaatttaattgaatttaacatgcaccatccatgaaaggccccagaaacaaacccccagtcttgagtttcatcagtgacttcagtgatccagggtttggcccagcccccggttttgcgcagtacagtgacctccttccacattttatcttcaatgaaaattgggaacatgtgtggtgatggcttgaatgtcaatgtcttctctcaattttgctatctgaagtttctttttttttttttgtcttcagacggagtcttcctgtgttgcccaggctggagtgcagtgtcacgatcacagctcactgcaacctctgcctcccaggttcaagtgattctcctgcctcagcctcccgagtacctgaaactacgggcgcatgccaccatgcctggctatttttttttatttttagtagagactgggtttcaccgtgttagccaggatggtgctgatcttctgaccttgtgatatgcccacctcagtttctgaaagtcctgggattacaggcataagccactgcgactggcctgctaatttgttttaagagaggcagagatgtagggaaataaggacagagacagataagtaacaaagataatgagactgagaaacagagttagggacaaagagggaacacatagagatagagatggggagccagtggcagaaagacagagggcaaacctcagacagtatacagagaggaagagagagagacacagagagagagagagtcacgtaaggagaaggaggaggaggaggaataagaggagagagagtaggaaaaggaaggaggaggggagaaaggagggaaaggagagaaacagaataagagataggaaaaagagagagtgacataaaaagagaggaagaaagaatgaacaagacaaataaggtatctttagagggagatgaaaagaaagaatgagagagaaagagattggggagaaatcagattcaaatagatggagataggaagttaagcaagatggaagaaagccaaaaaaaggagaggaaaagaagaaaagctgtgtctgacaggtatagacagagaaaaagacaggagtagggctgccaggggcagaaagaaaggtataacccaagccaaaagggtactgcagccaaagaaattcgaaaggtgtccaaacagacggccccagagatggggagggcgtgatgagagggagataataagcctcaggctgttgtgaaaaatgttaggtcacacaaagagttacagaagaacaggcccagagacctgcctgtttctaagctcatgtcacccaccttctgggtatgccaaagggatgtggacacttttccaaacacctccacatagacacacttgtccaacaacttgacaggcatagggaatgcctgaaaactcacacttgacatggcctttccaaggtttgcagattatgaaacactgaagtgaagggaaaggctccctttgtctctgcctgagctttttccagcaccctttgttcttatttttctcccaatctgtggtttagaatctactgggggttccttgcacctctgagaatcagtggaagccatagaccctccctgtctcatacgcatcagtcccattcacaatttatatacaattgggcttttccccccttgagctcttgaagcctgtggatctcaggctgagactagaagacagcctagagacacagacacagacacatggccagaaacacataccccacacataccccagaaacaaacacaaggcagtaaccaaaccttaagcctccttagcacaagccaagtgctattctaagaatttttacaattgtctcctttaacctcacaagagccctttgagggaggtgccattctccccatatgagaagtgaagaacactaagaatgttgtcataaatgtgctcacactcacacatgtacaggtacacacacacacacacacacacagaaccagaaatgggtcattatactctgtgggtttcaccaaccctttggaactgtaacagaagctagaaactctcttatataaaaatgaacatggcgccgggtgcagtggctcatgcctgtaatcccactttgggaggccaaggcaggtggattatctgaggtcaggagctcgaggctagcctggccaatatgatgataccccatgtctactaaaaatagaagaaattagccaggcgtggtggcaggtgcctgtaatcccagctacttggtaggctgaggcacaagaatcatttgaaccacctgggaggtggaggttgcagtgagccaagatggtgccactgtgctccagtctgggtgacaaagagagactccatctcaaaaaaaaaaaaaaaaaaaaaaaaaaaaaaaaaaagagagagagagaaatgaacgtggcacatccactcaaagatttgcatctggtttcagagcagcttcctaaaagtccaccctgaattgtaggttaaaggccagtcttatgcaaatctgttgcagtggacatttgtgtctgggcttagggacatggcagagcgaagtgtatgcacctgccctgtgcccacactggtgaccttctgtgtccacagggaagcggatttgtcttggtgaaggcatcgcccgtgcggaattgttcctcttcttcaccaccatcctccagaacttctccatggccagccccgtggccccagaagacatcgatctgacaccccaggagtgtggtgtgggcaaaatacccccaacataccagatccgcttcctgccccgctgaaggggctgagggaagggggtcaaaggattccagggtcattcagtgtccccgcctctgtagacaatggctctgactccccgcaacttcctgcctctgagagacctgctacaagccagcttccttcccctccatggcaccagttgtctgaggtcacattgcaagtgagtgcaggagtgagattatcgaaaattataatatacaaaatcatatatatatatatgttcttgttttttgagacagagtctcacactgttgcccaggctggagtgcagtggcgtgatctcggctcactgcaacctccacccccggggatcaagcaactctcctgcctcagcctccctagtagctgggattacaggcatgcactaccacgcttggctaatttttgtatttttagtagagatggggtttcactgtgtaggccaggctggtctcgaactcctgaactcaagtgattcacccaccttagcctcccaaagtgctgggattacaggcgtgagtcaccgtgcccagccatgtatatatataattttaaaaattaagctgaaattcacataacataaaattagctgttttaaagtgtaaaatttagtggcgtgtggttcattcacaaagctgtacaaccaccaccatctagttccaaacattttctttttttctgagatggagtctcactctgtcacccaggttcgagttcagtggtgccatctctgtccactgcaacctccacatcctgggttcaagtgattctcctgcctcagcctctggaggagctggtatcacaggcgtcccccaccacgcctggctaaattttgtatttttaggtggtcttgaactcctgatgtcaggtgattctcctagctccaaatgttttcattatctctcccccaacaaaacccatacctatcaagctgtcactccccataccccattctctttttcatctcggcccctgtcaatctggtttttgtcactatggacttaccaattctgaatatttcccataaacagaatcatacaatatttgattttttttttttttttgaaactaagccttgctctgtctcccaggctggagtgctatggtgcaatttttgttcactgcaacctctgccttccaagatcaagagattctccagtctcagctcccaagtagctgggattacaggcatgtactaccatgcctggctaattttcttgtagttttagtagggacatgttggccaggctggtggtgagctcctggcctcaggtgatccacccacctcagtgttccaaagtgctgatattacaggcataatatgtgatcttttgtgtctggttgctttcatgttgaatgctatttttgaggttcatgcctgttgtagaccacagtcacacactgctgtagtcttccccagtcctcattcccagctgcctcttcctactgcttccgtctatcaaaaagcccccttggcccaggttccctgagctgtgggattctgcactggtgctttggattccctgatatgttccttcaaatctgctgagaattaaataaacatctctaaagcctgacctccccacgtcaagaggtgatctgtgccattttgtgtgtgattcttttattgtcgggtctctagggatttttctggaaggaatgttggtgagaatgcctctctcacctcaatgccaactctgtgaagggccaaaccattgtcttgctcatccctgtactctcaacacagcgtgtggcatatgacaggtgttcaaaatatttggtgaggaatgaatgaatgagtggctaaatcagccaccccctacccccacagcccacccaaaatggagctaggcctcctccatcagactatatctcctccatatcccactttcttgtgaggtccagaataaattctcatccccaaagggcccaggatgcccccattctgcctaccagttactctcgataccccgtgctgcaatgccaccttttgataaagcttagcgctattcttgcagtggaaccactctaaatccagcctccaggctggacgcggtggctcatgcctataatcccagcactttgggaggccgacgcgggtggatcacttcaagtcaggggttcgagaccagcctgaccaacatacaaaaaccgtgtttcaactaaaaaagatacaacaattacccgggcatggtggtgtgcatctgtagtcccatctactcgggaggctgagacagggtgatcgcttgaatgcaggagggagaggttgcagtgagccaagttcccgccactgcactccagcctgggtgacaaagtgagactccgtttcataaataaataaataaataaatccagcctccaaacattccaccagcaggaccaccacccttccctgagggaggtgctgcaagtttaatatcattatcacaaggatacatcagccagcctgtggccagtgggaagctggccctgggactccaccctgagatgctaaccttttggcgtctgcagcatctgccatctttgggctccttgtagcctctgaagagttaacagagactctgccagtacaaatgcatggcacacattccctcaccaaccacagaaataacaaacagagcaacatgtcttttaagagcgacagcagaatggaaggcgggaagaatgaggaccacggtgccctcagccttctcactcctcattgtccccagtggcctcttgctactctggttctggggccatcccacctcccagggctgcctctctcctaggccctggcctctgcccttcctgggggagcattttgcaaataaaccacagggctttctgaagtctgtccaggtggtgagatgggagagtgtggaagggatggaaaaggaggaggtgggtagacgggaaacagctcacaggcagcaaggcagttggggtcgtactttgggtgggattccacagacaagcttgggaattcctttctggcttatatattaactacagaagcttctggaattttataaggtgaaatggagaggagacagactggagggtgaaattctgatagacttgaggctttgagatgtggtcctggggtggagcaagacaagaaaagtactggagattggggtttgaggagtctatgcaattatttttatttttaaaaatctttgtggctacatagcaggtgtatatatttatgtggtaagtgagatatttcgatacagacatacaatgtataatcacaggcatacaatgtagacaggcataaagtgtatagtcacatataataataacatcatggtaaatggggtaaccatcacctcaagcactcatcatttctttgtgttacattagagttatattccctctgttatgctaaaatgtaccacaggctgggcacagtggttcatgcctataatcccagtattttgggaggctgaggcagacagattgcctgagctgaggagttcgaaaccaccctgggcaacatggtgaaaccctgtctctacaaaaatacaaaaaaaaattagccaggcatggtggcatgcatctgtaatcccagctactctggaggctgaagcgggagaattgcttgagcctggtaggtggagtttgcagtaagccaagattgtgccactgcagtcccgcttgggtgacagagtgagactccatctcaaaaaaataaacaagcaaacaaacaaaagtacaatgaattattgctgacggccatcaccttattgtgctatcaaaaactagatcttattcattgtatcgaactctatttttgtacccattagccatccccagtccctcacctccacccttcccagtctctggtaaccatcattctactctatctccatgagttaaattgtcataatttttagctcccacgaatgagtgagaatgtgcaaagtttgcctttccgtgcctggcttatttcacctaacataatgtcctgcagttccatccatgttgctgcaaatagcaggatatcattcttttttaatggttgaatagtactcccttctgtatgggcaccatatgaacacttaggttgattccataacttggctcttgcaaacagagctgcaaaatacactggagtgcagatatctcttcagtatacggatttccttttttttgatatagatttagcggtgggattgctggattgtgtggtagctctatttttagtggttttctttctttttgtttgtttattttatttctttattcattttttttgagatggagtcttactctgtcacccaggctggagtgcagtggtatgatctcagctcactgcagcctccacctcctgggttcaagcgattctcctgcctcagcatcctgagtagctgggactacaggcacctgccgacacgcttggatgatttttgtaattttagtagagatggagtttcaccacgttggccaggctggtcttgaactcctgacctcagatgactcgcccacctcagcctcccaaagtgctgggattacaggcatgagccaccgcacctggcctattctacttttttttttctttttcttagggaaactccatgttgttcctcaaagtggctctattaatttacattaccaccaacagtgtacaagggttccatggggattacttcagattgagtggtcagagagggctcctttgagaagacttctgagaattggccatgggaaggtgtggggagaagccttctcagctgagggaacagcaaggtcaaagacccagaggtaagaaagcaagcttggaaccttccaggagcaacaaggcattggctctgataatgccttctcctgctaaaatgacagctctaggaaggcagggttcttatttcctgtctatctatctatctatctatctatctatctatctatctatctatcatctatctatctatcatatatctatctatccgtctgtctttatttatttatagaggcagagtcttgctctattgtccattgtattagtccattttcatgctgctgataaagacataccgtagactgggtaatttataaagaaaaaaagtttaataaactcacagttccacatggctgcggaggcctcacaatcatgctgaaaggcaaaagttgcgtcttacatggtggctggcaagagagagaatgagaaccaagagaacggggtttccccttataaaaccaacagatctcatgagacttattcactactatgagaacagtatgggggaaaccacccctatgattcaatgatcacccaccgggtccctcccacaacacgtggggattatgggagctgcaattctagatgagatttgggtggggacacagccaaaccatatcaccaaggctggagtgcagtggtatgatcatggctcattgcagcctccaacttctgggttcaagcaatcctcccacctcagcctcctgattagtggtgactacaggtgtgtgacaccacctctggctggcttttaaattttttggagagatggagtcttgctattttgcccaggctggtctcaaactcttggtctcaaatgatcttcccatcttgacttcccaaagcactgagattacaggtgtgagccattgcacctggctggttctcatttatttaagtattactgctgtcatggtttgctttggtccctgtgggttcccccaccctttgaaacaagtgcctgacacatggtagggttcacaataaatatttgtagaataaataagagctgagccttcaaggggtccaaaatacttgcacggagtttttctaggtgggcacgatgccacccagctgcaggactatttcagggtgttcattggctcctctggttgatcttttgatgcctgtataagaactcccacttctccaaatctgggggccacatctttagagttctcagctttctgtctctcaggaatgaaggagataatatcactgattggagccagattgtctggatgttgaatcccagctccttgctggtgcctttgagaagtgactttagccctccatgtgccttatttctgcctctgtaaacggagatcatcatagcacctctcatgtggggctgtttgacgattcggtgaggtatgtgcaaagagctttttgcacttgcaaaaagtgtctgggatatcattgtgtgctgtaagaatgagctgtaatgattatcactgagacttgttgttgggattaaaagagatcaggtctgtcagggcctggtacacaagaggtactagaaaaataatgggaggtttaggtgctccttcctccagatacactcgtaaatatgactgctgtgtcgtcatctactttttagggatatgaagaatctgctgtgtgaccttagatgaataaatctctgattcactctgggccttggttttccctgcctctgaaaggggtatgatgtttccttcccttttgcctgggcgcaggtactgggtgctttgtagttaagggagtatcagctggtggctcaagggtagattctaacaggaacttccaagctggagaaatactaggctacacctcagagaggggaggtgggtaagggaggcccctgctggtgtgagcaggaggtgggtgagggaggcccctgctggtgtgagctcgatcctttatccctgagcttcagactctcttaagccttcccacatgagaaactgacatccagatggaggtggggaccctgcaatgtaaagccaggtcacctgtccacagaggtgtgtctgataaccagggcatgataggttagagagtgtggtggctaaaagcaccagcccttggatcacctgaggtcaggagttcaagacctgcctggccaacatggtgaaactccatctctactaaacatacaaaaattagcttggtgtggtggtgggcacctgtaatcccagctacttgggaggccgaagcaggagaatcacttgaacccaggaggcagaggttgcagtgagctgagattgcgccatttcactccagcctgggtgacaaaagcgaaactctgtctcaaaaaaagcaccagccttagactcagagagaccaggatttgagcttgggggacctcagcttcctctctgtgtgacctcaggtaagccacttatactctttgggcctcagtttcctcatttgtaaaattgaggccgtgattgaacctccttcaagagctgttgtgagtgctataggggatactatcttaaagcacttaatgcagtgcctggcacatggtagttctgtagaagtgcttgctattttaattcacgtgcacaggtcctgcctgtgggtgagggctcagtagatggaacagcgatggttgatatagttgggattgtgaggattattatgacaggggctaatttgaatgggcctgtgttgtctgccctccccactgccagcctgatttccctctgcctggctttgcagctcctcccattggctgctggggtgtaccccttgcatgcggatgagaaacgagtcaatgaagtcccgtggggaattgggatccagcgtgcactggttgtgctccactttcttggcaatgaagtcctccagcccttgcagctccttaaaggcctgttgctgtggtcctggcaggtgtttcatcaccgaagagaacatctcatagagctggggttgcagagagaggatgggaagggaaggaccgctgtcaggaggcaggaactgatgggaatgggacctgtgtcagagcaagaaaggcactgggttaaaggcacctacctgcccaggtgtttaggtatttcagtggggctggatgggaacatatatgtaagtcttcatgagggttaagtgagaaggctactgtccagaaatttaggtaacatggactgaggcacacatccaggtttcaatgaaggtggattggagcgcacagctaggtgttcagacattcaaggcaggatgtgttggaaaacacctgttcaactgtttagctactcagacggggctggtatgaggggagtgtcacctgtctaggtatgtaggcattcaggtgggcctggttgaagacaactaccgagatacttattgggtctggattgggggcttttgttcagatgttcaggtattaaagtggggctggtgaagatgttgaagaggaggggatacctgttgaggtgtttacctatccaagtgggatgcactgggggcacctctgtaaggtgtgtgattggaaagggttggggaacacttatttgggtgtctggggaataatctgtctagatatttatgtgtttagggtattggttagggcacctgtccatgttttcaggtatttaagtgggtgaagtagagggcgcttggccagatattccagtgggctaatctgggacatttgtgtagatgtgaaatgattatgatgggctggattttgcagcaccaggtgttcaggtatgcaggaggcgggttgaggcagtcacttgtccaggtgttaaaatattcagaagaactgggtagggagcatctgttagaaattatggtaagttggggatgggaacatgtgcccaggtgagagagctgagctgagggtatgcatcctgccttcaggctctgttttggggatcatctgttgagctatccaggtgtccttggagactgggtattgggcacccatcctgggttctggtgcaactttccggttgtccaatattgggggctgattttgaggggacactgtctggaggggggtgggagtttggggcacctgtccccatgtaggggagcagttggcaggttgtggtaggggcgtgacagccaggctgcagccggttacctgccccgtggagggtgccgtgaactggaagctttccagcatcatgcacagcagtgacaggaacttttgtcctcatagtcaaagcggtccccaaagacaatggagctgatgacattggaggtggtacggcttaggaagaaggtgggatagatgctggagcttgcgggtgtggcggtgtattgggggagaagggggttgggaagagagtcaactcagagctctgatgctagtctgagaattccaggaggcactgccctgatgagccacattcccagcgccagactacagggctgggagcgcgggcgcctttccccaccaaggcccggtccccagacagaacgcgcgcgggttcctcgccctgggcgttttccttctcccgcctccacactcggggtgttctgctcaccgcgcgtgccctagacggccttgatgaggaagcccgccttctcctggatacctccttgatgccgcgcttgcccaccccgaagtcccgcagggtggcgatggagaagcgcctgagcggcttggtgcgctcccaggtgcggcaggtcactcctgggagaggaacgcaggggtggggagaggtcaggccgtgggaacaggcacagaagcctcgcgcaggagaactgcaaactcagtcagagaaacacagagagaaacagagaccgaacaggaacaagaggatcagaaaagagacaggccagacagacagatgcacagcagaacagagggagacgggacactccatggagcatagggagggaaggagggagagaaaaagacagagatgggagaagatgatggaagggggggaagtgaggtatggggagatctgcgaggagaaaataaaaatggtgaaagagtcttttttgtttgtatgtttgtttatttgtttttgagacggattctcgctctgtcgcccaggctgaagtgcattgtcgccatcttggccgctcgctgcaacctccaccttccaggttcaagcgtaaaaatggtgaaagactcctaagctgagctggtgaagacagaaaactacggaaagagaaacagagacggtcgggaaggaggagagactgttatcaggagaggaggctggagatgtggaagtaggaataccgcgaaatcctaagacagaggaagaagaaggagaggtcagtggacagaggagaaactcagagaagcacagaagctgagaaggacaaaaagacaaatgaagacagagaaccagggttggaaaacagaaacccaggattgtcctcagagatggggaaagaggagactccagtgagagaggcagggagaaatctggacagggacagggacagggatgctggagacaggcaagagggagaggccgcaatgaagggagctggggagcgaaggccagacagggggctctgccgcagccttcagtgggcaggagagtcagggagaaggctggggacacggaggccatcgagtcggcctggccaccttccccctcttgggcaccccctcaccatagcctttgaagagccagtcgaaggtggcctgctcgcctcgcccgctgaactcctcagcctggtccaccagagcctccttgacggcatcatgtccgcacagcaccacgacccgccggggccccaggtgaatggtgaacacggggccatagcgctcactgatctgatggaggcaggtgggagtggttagagggagcagcccccactctgaatgggcgcagcaccgagatatcatgtactgggatgttttgcccccaggttctcacagtcagggagctggacatcccaagatcctgtctttccagcctaggaccctgatgctgaaactccaaaactccctttcctaagactctggtccacactggtcaatcccctgccacaaagcctcagccaactgggcagcccccaccccgtgccacccatctccctgccttgggacaccttcatgatggagtcacatatgtgctctgtgttcagctgcaggtagtttccaataaagagcaatggggtgggtcccaaaggcagcttccccctgctcttcctctgctgccagactgacatcaagaccatcacagtcaggctggccagcaaggccaccagaagcagccctgaggccagcatggtggcagtggaatgatagatggtgacggctggggtggtttgcctttatactgcctgaaaaaaaggggtggactttggctggttataaaatcacctcatttcacctcccaactacatcctctccgtgaaccccacctagctttggacacaaccagcaaaggaggaggagggggaccccagggaagctgaacagagagggtacctcccgactaaatctgtggtacctcaggaggggtgccccagggctgtggatttaggagcgggcaacagataagctgtagaacaaaggagttgggaatatttgcataggggagcacttgggctttggatttgggatctgggagtgagaatgcaccccaaaattgtggatttgggggttccgaggagaagaatgcaaaggtctgggttttattagggtgagatggaagtgtggctgtgcatctgggggtcttctgttgtggaggatgcagggttaagggtctcaggaggggggattccaggcatatggacttgagagtcttggggctaggaagacccagggctgtgggtgtggagtttctgggaggaggagtgtgtagcaagcccagtaggtcgtcccagaaagcccagcagcccagcactgggcccaggggctgacttgattttgcttttgcccagagctgtctcttgatgcccggaatcctcactggaacctcagagtgaggccaggatgggtgccatggaaactccactgaccatctctggttggcaaaggggtggaagaccagagccatatttcaagggaaatttccacacacctggagaagagtaccttgtgttatcagttggcagagggattggccaggtctggagaaatagtaacaacaacccccaaatggtggaaaaagctcaagtgtcccaagggttgtaatttacaaagacctccactggtttccaacccagatccttagggaaacccactagattgattgtatcatccctattaatattatgattgttgtttatctttctcattttatataagggtctacagaggacagaaaacagacagtaggcaggggacatgcatgagtggccctgaactcccatctctcctgctctgagtacttagttcccctttctggatttggttttcccaccagtagaaagggagtatttaggaaaagatcatatttgagggtgagttaagtctcctttaagggcgattcctgctttgtcaggggctactactattagcaggcagagggaacaatcgcagaagagattggggtaaactccagtgttaggcacagaggtagatcctggggtgggtgtgagtggagaagggatgagtagccctcctgtccttccctcagggaaattcttccttagtaaacggggcagttaccctttggtgagcatggtcgtatatgggaagattaagtctaggtgtgtatgatctggtcgaatctttgtaacagtgaggtttgttcatcatgatcatcctcattttgaagatgagcagatctagactccataaatcctcttgagtgaggtcacacagggaataagtgagggagctgggatgtgaactttcttctcatcccccgtcattgactggcctagatcaccctgggagtcaagtgcaggccaggcttagcaggaggtgagtgctgagactggcatcagcagtggacacactgatgacattggcgggttctgatatcagcatcaggtgtgttcagggagggaaaggcatcgtgttggctttgggactgcccccaggtgtatgcgaggatgggaaacaagttggtactgaacttgataccaggagaatattgaagtcatgtttggtgccaagagggctctgtgtgttgtttcaggtttcattgagggcataagtgtgtcctgcttcaggagggcgtaagaatggcaccatatagggtctgaggttggtcccagtgggctgtgacatgagcatgaggcaggtgctgatattggtcctatgtgggtctgagatgagcctcagccagatc

SEQ ID NO: 13 - DNA, 1690 bp, Mouse
gaagcattgaggaggatcacacacacagttgtagggagaacacagagaagtaaattgctgacaaacaagcagggatggacctggtttcagctctctcactggaaacctgggtgctcctagcaatcagcttggtgctcctctaccgatatgggactcgtaaacatgaactttttaagaaacagggaattcctgggcccaaacctctgccatttttaggcactgtgctgaattattacaagggtttatggaaattcgacatggagtgctataaaaagtatggaaaaacatgggggttgtttgatggtcaaacgcctctccttgctgtcacagacccagagacgattaagaatgtgctagtgaaggaatgtttttctgtcttcacaaaccggcgggattttggcccagtggggataatgagtaaagctatctcaatatctaaggatgatgaatggaagagatatagagctttgctgtcccccacattcaccagtggaaaactcaaggagatgttccctgtcattgaacagtatggagacattttggtaaagtacttgaggcagaaggcaaagaaaggcaagcctgttactatgaaagatgtgttaggtgcttacagcatggatgtgatcaccagcacatcatttggagtgaacgtggattccctcaacaacccagaggatccttttgtggagaaagccaaaaagcttttaagatttgatttttttgatcctttgctcttctcagtagtactttttccattcctgacaccagtatatgagatgttaaatatctgcatgttcccaaaggattcaatagaatttttcaaaaaatttgtggacagaatgaaggaaagccgcctggattctaagcagaagcaccgagtggattttcttcagctgatgatgaattctcataataattccaaagacaaagtctctcataaagccctttctgacatggagatcacagcccagtcaattatctttatttttgctgggtatgaaaccaccagtagcacactttccttcaccctgcattccttggccactcaccctgatatccagaaaaaactgcaggatgagatcgatgaggctctgcccaacaaggcacctcccacgtatgatactgtgatggagatggaatacctggatatggtgcttaatgaaaccctcagattatatcccattgctaatagacttgagagagtctgtaagaaagatgttgaactcaatggtgtgtatatccccaaagggtcaacagtgatgattccatcttatgctcttcaccatgacccacagcactggtcagagcctgaagaattccaacctgaaaggttcagcaaggagaacaagggcagcattgatccttatgtatatctgccctttgggaatggacccaggaactgccttggcatgaggtttgctctcatgaatatgaaacttgctctcactaaaattatgcagaacttctccttccagccttgtaaggaaacacagatacctctgaaattaagcagacaaggacttcttcaaccagaaaaacccattgttctaaaggttgtgccacgggatgcagtcataactggagcatgagtctccctcaaggagttcttctgagttcttcagaaaggcagtgtctaagaacatcggacattttagtttcatcatgagtaaaattgagatgaataaggggc

SEQ ID NO: 14 - DNA, 2768 bp, Human
cactgctgtgcagggcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctatatggaacccattcacatggactttttaagaagcttggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctctgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagtattatcagatgctttcctgcacattaaggagaaatctatagaactgaatgagaaccaacaagtaaatatttttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaataaaaaacagtcgattggtc

SEQ ID NO: 15 - DNA, 39 bp, Artificial Sequence
  Primer
ccggaattcaggaaagacatgatactgtcggcagaagcc

SEQ ID NO: 16 - DNA, 41 bp, Artificial Sequence
  Primer
cgcggatccggccgctgcaggcgcagaactggtaggtatgg

SEQ ID NO: 17 - DNA, 21 bp, Artificial Sequence
  Primer
gatgtgctccaggctaaagtt

SEQ ID NO: 18 - DNA, 21 bp, Artificial Sequence
  Primer
agaaacggaatgttgtggagt

SEQ ID NO: 19 - DNA, 28 bp, Artificial Sequence
  Primer
ccgcctctagagcggtttcttaccaata

SEQ ID NO: 20 - DNA, 30 bp, Artificial Sequence
  Primer
ccgccggatccagccagagtaggcaaatct

SEQ ID NO: 21 - DNA, 20 bp, Artificial Sequence
  Primer
ggatgaatatgccctacatg

SEQ ID NO: 22 - DNA, 20 bp, Artificial Sequence
  Primer
tgatgggcagcaggtctcat

SEQ ID NO: 23 - DNA, 20 bp, Artificial Sequence
  Primer
acagaaactatgtgagcctg

SEQ ID NO: 24 - DNA, 20 bp, Artificial Sequence
  Primer
atgggacggttcacatgttc

SEQ ID NO: 25 - DNA, 1450 bp, Human
gtgagcttgctccttaagttacaggaactctccttataatagacacttcattttcctagtccatccctcatgaaaaatgactgaccactgctgggcagcaggagggatgataatcctaactccaatcactggcaactcctgagatcagaggaaaaccagcaacagcgtgggagtttggggagaggcattccataccagattctgtggcctgcaggtgacatgctgcctaagagaagcaggagtctgtgacagccaccccaacacgtgacgtcatggccagtagggaagatgagctgaggaactgtgtggtatgtggggaccaagccacaggctaccactttaatgcgctgacttgtgagggctgcaagggtttcttcaggagaacagtcagcaaaagcattggtcccacctgcccctttgctggaagctgtgaagtcagcaagactcagaggcgccactgcccagcctgcaggttgcagaagtgcttagatgctggcatgaggaaagacatgatactgtcggcagaagccctggcattgcggcgagcaaagcaggcccagcggcgggcacagcaaacacctgtgcaactgagtaaggagcaagaagagctgatccggacactcctgggggcccacacccgccacatgggcaccatgtttgaacagtttgtgcagtttaggcctccagctcatctgttcatccatcaccagcccttgcccaccctggcccctgtgctgcctctggtcacacacttcgcagacatcaacactttcatggtactgcaagtcatcaagtttactaaggacctgcccgtcttccgttccctgcccattgaagaccagatctcccttctcaagggagcagctgtggaaatctgtcacatcgtactcaataccactttctgtctccaaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatgccgctgctccaggagatctgcagctgaggccatgctcacttccttccccagctcacctggaacaccctggatacactggagtgggaaaatgctgggaccaaagattgggccgggttcaaagggagcccagtggttgcaatgaaagactaaagcaaaac

SEQ ID NO: 26 - DNA, 1350 bp, Mouse
cttgttttccaggcactgaggaccgcagtccctaattcctggcagttcctgagatctcaaggaaagcagggtcagcgaggaggcctggggagaggaggcatcctacacccaatcttgtggcctgctgcctaagggaaacaggagaccatgacagctatgctaacactagaaaccatggccagtgaagaagaatatgggccgaggaactgtgtggtgtgtggagaccgggccacaggctatcatttccacgccctgacttgtgagggctgcaagggcttcttcagacgaacagtcagcaaaaccattggtcccatctgtccgtttgctggaaggtgtgaggtcagcaaggcccagagacgccactgtccagcctgcaggttgcagaagtgtctaaatgttggcatgaggaaagacatgatactgtcagcagaagccctggcattgcggcgagccagacaggcacagcggcgggcagagaaagcatctttgcaactgaatcagcagcagaaagaactggtccagatcctcctgggggcccacactcgccatgtgggcccattgtttgaccagtttgtgcagttcaagcctccagcctatctgttcatgcatcaccggcctttccagcctcggggccccgtgttgcctctgctcacacactttgcagatatcaacacgtttatggtgcaacagatcatcaagttcaccaaggatctgccgctcttccggtccctaaccatggaggaccagatctcccttctcaagggagcggctgtggaaatattgcatatctcactcaacactacgttctgtcttcaaacagagaatttcttctgtgggcctctttgctacaagatggaggacgcagtccatgcagggttccagtacgagtttttggagtcgatcctccacttccataaaaacctgaaaggactgcatctccaggagcctgagtatgtgctcatggctgccacggccctcttctcccctgacagacccggagttacccaaagagaagagatagatcagctacaagaggagatggcgctgattctgaacaaccacattatggaacaacagtctcggctccaaagtcggtttctgtatgcaaagctgatgggcctgctggctgacctccggagtataaacaatgcatactcctatgaacttcagcgcttggaggaactgtctgctatgacgccgctgctcggggagatttgcagttgaggcccaggcttgcatcctttccccagacccccagggatacactggcctggaaagggtacagcgctggaccccacacgggaaccagcaggaaggagcttgggagtggcaatgaaatgctgaacagtc

SEQ ID NO: 27 - protein, 286 aa, Mouse
MTAMLTLETMASEEEYGPRNCVVCGDRATGYHFHALTCEGCKGFFRRTVSKTIGPICPFAGRCEVSKAQRRHCPACRLQKCLNVGMRKDMILSAEALALRRARQAQRRAEKASLQLNQQQKELVQILLGAHTRHVGPLFDQFVQFKPPAYLFMHHRPFQPRGPVLPLLTHFADINTFMVQQIIKFTKDLPLFRSLTMEDQISLLKGAAVEILHISLNTTFCLQTENFFCGPLCYKMEDAVHAGFQYEFLESILHFHKNLKGLHLQEPEYVLMAATALFSPGFCMQS

(19) United States
(12) Patent Application Publication
(10) Pub. No.: US 2006/0288436 A1
(43) Pub. Date: Dec. 21, 2006

(54) SCREENING SYSTEMS AND METHODS FOR IDENTIFYING MODULATORS OF XENOBIOTICMETABOLISM

(76) Inventors: David D. Moore, Bellaire, TX (US); Ping Wei, Houston, TX (US); Steven S. Chua, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 11/464,479

(22) Filed: Aug. 14, 2006

Related U.S. Application Data
(63) Continuation of application No. 10/380,555, filed on Jul. 25, 2003, now abandoned.
(63) Continuation of application No. 10/219,590, filed on Aug. 15, 2002.
Application No. 10/219,590 is a continuation of application No. 09/666,250, filed on Sep. 21, 2000, now abandoned.
(63) Continuation of application No. 10/268,822, filed on Oct. 10, 2002.
Application No. 10/268,822 is a continuation-in-part of application No. 10/219,590, filed on Aug. 15, 2002.
Application No. 10/219,590 is a continuation of application No. 09/666,250, filed on Sep. 21, 2000, now abandoned.

(51) Int. Cl. A01K 67/027

(52) U.S. Cl. 800/18

(57) ABSTRACT

The present invention provides mice having reduced CAR receptor activity and mice expressing a human CAR receptor. These mice are useful in screening methods to identify compounds that modulate CAR receptor activity, compounds likely to have CAR-mediated toxicity, and analogs of these compounds with less potential toxicity.

### CROSS-REFERENCE TO RELATED APPLICATIONS

This application is a Continuation of application Ser. No. 10/380,555 filed on Jul. 25, 2003; which is a U.S. National Stage application of PCT/US01/29672 filed on Sep. 21, 2001, now expired; which claims priority to Ser. No. 09/666,250 filed Sep. 21, 2000, now abandoned. This application is also a Continuation of application Ser. No. 10/219,590 filed Aug. 15, 2002; which is a Continuation of application Ser. No. 09/666,250 filed Sep. 21, 2000, now abandoned. This application is also a Continuation of application Ser. No. 10/268,822 filed Oct. 10, 2002; which is a Continuation in part of application Ser. No. 10/219,590 filed Aug. 15, 2002; which is a Continuation of application Ser. No. 09/666,250 filed September 2000, now abandoned.

### STATEMENT REGARDING FEDERALLY SPONSORED RESEARCH OR DEVELOPMENT

Statement as to Federally Sponsored Research This invention was made with government support under NIH grant NIDDK RO1 DK46546. The government therefore has certain rights in the invention.

### REFERENCE TO A COMPACT DISK APPENDIX

Not applicable.

### TECHNICAL FIELD

In general, the invention involves screening methods for identifying modulators of metabolism of any of a wide range of foreign compounds, collectively termed xenobiotics.

### BACKGROUND OF THE INVENTION

A number of cytochrome P450 (CYP) enzymes able to metabolize diverse substrates serve as a primary defense against potentially deleterious effects of xenobiotic compounds. Induction of the expression of individual CYP genes in response to particular xenobiotics is a central component of this metabolic mechanism. One of the best characterized of these responses is the induction of specific CYP genes by a diverse group of agents known as “phenobarbital-like” inducers. Exposure of animals to any of a chemically diverse series of compounds exemplified by phenobarbital (PB) results in a potent activation of expression of a specific subset of CYP enzymes and other proteins associated with xenobiotic metabolism. In the mouse, these PB-like inducers increase expression of CYP2B10 and several other genes. The pesticide contaminant 1,4-bis[2-(3,5-dichloropyridyloxy)]benzene, referred to as TCPOBOP, is generally considered to be the most potent of this group of inducers.

Xenobiotics, such as therapeutic drugs, insecticides, polycyclic hydrocarbons, and some natural products, are often metabolized via oxidation reactions catalyzed by CYP enzymes. These reactions add hydrophilic groups to xenobiotics, allowing the body to rid itself of these noxious or simply insoluble materials. For example, oxidation of polycyclic aromatics produces epoxides, which are very reactive electrophilic groups. Usually these epoxides are rapidly hydrolyzed into hydroxyl groups which are then coupled to other groups, producing compounds water-soluble enough to be excreted.

Unfortunately, the intermediate epoxides may also be released into the cell as highly reactive electrophiles, possibly reacting with negatively charged groups in DNA and causing changes in the DNA sequence. Reactive oxygen species generated during metabolism of cocaine by CYP enzymes in humans has been associated with mutagenesis and chromosome breakage.

CYP-mediated metabolism may also result in other undesired effects, such as the rapid degradation of a therapeutically active compound, lowering its half-life in vivo. Alternatively, CYP enzymes may convert a prodrug into an active drug at a faster than desired rate resulting in a toxic concentration of the active drug in vivo. Additionally, the activation of CYP enzymes by the administration of a therapeutically active compound or exposure to another foreign compound may result in faster metabolism of a second therapeutically active compound, reducing its effectiveness or increasing its toxicity.

Because of the potentially deleterious effects of compounds that activate CYP enzymes, improved methods are needed to determine which compounds activate CYP-mediated metabolism and, thus, might cause side-effects if administered to humans. These compounds may thereby be eliminated from drug development or chemically modified to generate related compounds with less ability to activate CYP enzymes.

### BRIEF SUMMARY OF THE INVENTION

The present invention provides screening systems and methods that facilitate the identification of compounds that activate or inhibit a CAR receptor. Such CAR receptor-activating compounds are potentially toxic when administered to a mammal alone or in combination with other compounds, and are therefore preferably excluded from candidate drugs or drug development programs. Similarly, compounds that inhibit a CAR receptor may be administered to a mammal to decrease the CAR-mediated metabolism of a therapeutically active compound, potentially decreasing side-effects and re-establishing the therapeutic half-life of the compound in vivo. Such a decrease of metabolic activity may also be useful to decrease production of toxic products from appropriate precursor compounds.

Accordingly, in a first aspect, the invention features a transgenic mouse expressing a human CAR receptor. In a related aspect, the invention features a mouse having a mutation that reduces CAR receptor activity.

The animals of the present invention may be used to determine whether a compound modulates the activity of a CAR receptor. In addition, methods are provided to determine whether the metabolism of a compound is regulated by modulation of the activity of a CAR receptor.

Accordingly, the invention also features a screening method for determining whether a compound activates a CAR receptor. This method involves administering a compound to a transgenic mouse expressing a human CAR receptor and measuring induction of a CAR target gene. The compound is determined to activate the CAR receptor if the compound mediates induction of the CAR target gene. In one preferred embodiment, a CAR receptor inverse agonist is also administered to the mouse expressing a human CAR receptor.

Preferably, the CAR receptor inverse agonist is clotrimazole.

In another aspect, the invention features a screening method for determining whether a compound inhibits a CAR receptor. This method involves administering the compound to a transgenic mouse expressing a human CAR receptor and measuring expression of a CAR target gene in the presence and absence of the compound. The compound is determined to inhibit the CAR receptor if the compound decreases the expression of the CAR target gene.

In one preferred embodiment, a CAR receptor agonist is also administered to the mouse expressing the human CAR receptor. Preferably, the CAR receptor agonist is a functional CAR receptor agonist that is specific for human CAR, and the agonist is administered after the compound is administered to the mouse.

In yet another aspect, the invention features a screening method for determining whether a compound modulates the activity of a CAR receptor.

This method involves administering the compound to a transgenic mouse expressing a human CAR receptor and measuring a physiological effect mediated by the administration of the compound. The compound is determined to modulate the activity of the CAR receptor if the magnitude of the physiological effect in the mouse expressing the human receptor differs from that in a mouse having a mutation that reduces CAR receptor activity. In preferred embodiments, the physiological effect is assayed by measuring the toxicity or activity mediated by the administration of the compound or by measuring the half-life of the compound. In other preferred embodiments, the toxicity or activity is mediated by a metabolite of the compound. Preferably, the difference between the magnitude of the physiological effect in the mouse expressing the human CAR receptor as compared to a mouse having reduced CAR receptor activity is at least 2, 5, 10, or 20-fold. In other preferred embodiments, the magnitude of the physiological effect in a mouse having reduced CAR activity is at least 10, 25, 50, or 75% smaller or larger than the magnitude of the effect in the mouse expressing the human CAR receptor.

In still another aspect, the invention features a screening method for determining whether the metabolism of a compound is regulated by modulation of the activity of a CAR receptor. This method involves administering the compound to a transgenic mouse expressing a human CAR receptor and measuring the rate of metabolism of the compound. The metabolism of the compound is determined to be regulated by modulation of the activity of the CAR receptor if the rate of metabolism is faster in the mouse expressing the human receptor than in a mouse having reduced CAR receptor activity. Preferably, the rate of metabolism is at least 2, 5, 10, or 20-fold faster in the mouse expressing the human CAR receptor than in the mouse having reduced CAR receptor activity. In preferred embodiments, the rate of metabolism is determined by measuring the toxicity or activity mediated by the administration of the compound, measuring the half-life of the compound, or measuring the serum level of a liver enzyme. Preferably, these measurements are performed at more than 1, 3, or 5 time points after administration of the compound.

In another aspect, the invention provides a screening method for determining whether the metabolism of a first compound is modulated by a second compound. This method involves administering the first compound in the presence and absence of the second compound to a transgenic mouse expressing a human CAR receptor. A physiological effect that is mediated by the administration of the first compound is measured in the presence and absence of the second compound. The second compound is determined to modulate the metabolism of the first compound if the second compound effects a change in the physiological effect mediated by the administration of the first compound. In preferred embodiments, the physiological effect is assayed by measuring the toxicity or activity mediated by the administration of the first compound or measuring the half-life of the first compound. In various preferred embodiments, the toxicity or activity is mediated by a metabolite of the first compound. In still another preferred embodiment, the physiological effect is assayed by measuring the half-life of the first compound in the presence and absence of the second compound. The second compound is determined to activate the metabolism of the first compound if the second compound decreases the half-life, or the second compound is determined to inhibit the metabolism of the first compound if the second compound increases the half-life.

Similar methods for determining whether a compound modulates the activity of a CAR receptor or the metabolism of another compound may also be performed using a mouse having a mutation that reduces CAR receptor activity. For example, the invention features a screening method for determining whether a compound activates a CAR receptor. This method involves administering a compound to a mouse having a mutation that reduces CAR receptor activity and measuring induction of a CAR target gene. The compound is determined to activate the CAR receptor if the induction is smaller in the mouse having reduced CAR receptor activity than in a mouse having wild-type CAR receptor activity. In a preferred embodiment, a CAR receptor inverse agonist to is also administered to the mouse having reduced CAR receptor activity. Preferably, the inverse agonist is androstanol.

In another aspect, the invention features a screening method for determining whether a compound inhibits a CAR receptor. This method involves administering the compound to a mouse having a mutation that reduces CAR receptor activity and measuring expression of a CAR target gene in the presence and absence of the compound. The compound is determined to inhibit the CAR receptor if the decrease in expression effected by the compound is smaller in the mouse having reduced CAR receptor activity than in a mouse having wild-type CAR receptor activity. In one preferred embodiment, a CAR receptor agonist is also administered to the mouse having reduced CAR receptor activity. Preferably, the CAR receptor agonist is TCPOBOP, and the TCPOBOP is administered after the compound.

In still another aspect, the invention features a screening method for determining whether a compound modulates the activity of a CAR receptor.

This method involves administering the compound to a mouse having a mutation that reduces CAR receptor activity and measuring a physiological effect mediated by the administration of the compound. The compound is determined to modulate the activity of the CAR receptor if the magnitude of the physiological effect in the mouse having reduced CAR receptor activity differs from that in a mouse having wild-type CAR receptor activity.

Preferably, the difference between the magnitude of the physiological effect in the mouse having reduced CAR receptor as compared to a mouse having wild-type CAR receptor activity is at least 2, 5, 10, or 20-fold. In other preferred embodiments, the magnitude of the physiological effect in the mouse having reduced CAR activity is at least 10, 25, 50, or 75% smaller or larger than the magnitude of the effect in a mouse having wild-type CAR receptor activity. In yet other preferred embodiments, the physiological effect is assayed by measuring the toxicity or activity mediated by the administration of the compound or measuring the half-life of the compound. In another preferred embodiment, the toxicity or activity is mediated by a metabolite of the compound.

In still another aspect, the invention provides a screening method for determining whether the metabolism of a compound is regulated by modulation of the activity of a CAR receptor. This method involves administering the compound to a mouse having a mutation that reduces CAR receptor activity and measuring the rate of metabolism of the compound. The metabolism of the compound is determined to be regulated by modulation of the activity of the CAR receptor if the rate of metabolism is slower in the mouse having reduced CAR receptor activity than in a mouse having wild-type CAR receptor activity. Preferably, the rate of metabolism is at least 2, 5, 10, or 20-fold slower in the mouse having reduced CAR receptor activity than in a mouse having wild-type CAR receptor activity.

In preferred embodiments, the rate of metabolism is determined by measuring the toxicity or activity mediated by the administration of the compound, measuring the half-life of the compound, or measuring the serum level of a liver enzyme. Preferably, these measurements are performed at more than 1, 3, or 5 time points after administration of the compound.

In yet another aspect, the invention features a screening method for determining whether the metabolism of a first compound is modulated by a second compound. This method involves administering the first compound in the presence and absence of the second compound to a mouse having a mutation that reduces CAR receptor activity. A physiological effect that is mediated by the administration of the first compound is measured in the presence and absence of the second compound. The second compound is determined to modulate the metabolism of the first compound if the change effected by the second compound in the physiological effect mediated by the administration of the first compound is smaller in the mouse having reduced CAR receptor activity than in a mouse having wild-type CAR receptor activity.

In preferred embodiments, the physiological effect is assayed by measuring the toxicity or activity mediated by the administration of the first compound or measuring the half-life of the first compound. In various preferred embodiments, the toxicity or activity is mediated by a metabolite of the first compound. In another preferred embodiment, the physiological effect is assayed by measuring the half-life of the first compound in the presence and absence of the second compound. The second compound is determined to activate the metabolism of the first compound if the decrease in the half-life effected by the second compound is smaller in the mouse having reduced CAR receptor activity than in a mouse having wild-type CAR receptor activity, or the second compound is determined to inhibit the metabolism of the first compound if the increase in the half-life effected by the second compound is smaller in the mouse having reduced CAR receptor activity than in a mouse having wild-type CAR receptor activity.

In preferred embodiments of various aspects of the invention, the mouse having a mutation that reduces CAR receptor activity is a transgenic animal. Preferably, the mutation that reduces CAR receptor activity substantially eliminates CAR receptor activity. In yet other preferred embodiments, the mouse having a mutation that reduces CAR receptor activity and the mouse having wild-type CAR receptor activity have the same genotype except for a mutation in the CAR receptor gene, promoter, or regulatory sequence. In still other preferred embodiments, the mouse having wild-type CAR receptor activity is a transgenic mouse expressing a human CAR receptor. Preferably, the mouse expressing a human CAR receptor does not express a substantially active murine CAR receptor or does not express any murine CAR receptor.

Preferred CAR target genes are murine CYP2B10 (SEQ ID NO: 11, Accession No. NM009998) and a transgene containing human CYP2B6 (SEQ ID NO: 12, GenBank Accession No. AC023172). Additional preferred CAR target genes include murine CYP3A11 (SEQ ID NO: 13, Accession No. NM07818) and a transgene containing human CYP3A4 (SEQ ID NO: 14, Accession No. A34101). Other possible CAR target genes include, but are not limited to, other CYP enzymes or other enzymes involved in xenobiotic metabolism. CAR target genes may also include a CAR responsive promoter operably-linked to a reporter gene, such as human growth hormone, secreted alkaline phosphatase, chloramphenicol acetyl transferase, luciferase, green fluorescent protein, CYP2B6, or any other reporter gene (see, for example, Ausubel et al., Current Protocols in Molecular Biology, Chapter 9, John Wiley & Sons, New York, 2000). Examples of appropriate promoters include native CYP promoters, such as the CYP2B10 promoter (GenBank Accession No. U48732; Honkakoski et al., J. Biol. Chem. 271, 9746-9753, 1996) containing the previously described phenobarbital response element (Honkakoski et al., Mol. Cell. Biol. 18: 5652-5658, 1998), the CYP2B6 promoter (GenBank Accession No. AC023172), the CYP3A11 promoter (Toide et al., Arch. Biochem. Biophys. 338 (1): 43-49, 1997), the CYP3A4 promoter (Accession No. AF185589), or synthetic promoter constructs in which DNA binding sites for CAR/RXR heterodimers are operably-linked to functional basal promoters (Tzameli et al., Mol. Cell. Biol. 20: 2951-2958, 2000).

In other preferred embodiments, at least one of the compounds tested in the screening methods of the invention is a member of a library of as few as 2 or 5 compounds to as many as 10, 20, 50, or more compounds, all of which are simultaneously administered to the mouse. Preferred routes of administration of the compounds include oral, intramuscular, intravenous, parenteral, intraarticular, intraperitoneal, subcutaneous, or any other suitable route.

Preferably, a compound that activates a CAR receptor or a compound whose metabolism is regulated by modulation of the activity of a CAR receptor is eliminated from drug development. If a first compound activates the metabolism of the second compound, then the first compound, the second compound, or both compounds are preferably eliminated from drug development. It is also contemplated that other animal models, such as a rat or other rodent having reduced CAR receptor activity or expressing a human CAR receptor, could be used in any of the various aspects of the invention.

By “CAR receptor activity” is meant CAR-mediated induction of a gene, denoted a “CAR target gene,” or a transgene operably-linked to a CAR responsive promoter. The level of induction of the CAR target gene or transgene may be determined using standard assays for measuring the level of encoded mRNA or protein (see for example, Ausubel et al., Current Protocols in Molecular Biology, John Wiley & Sons, New York, 2000). Alternatively, an enzymatic activity of a CAR target gene, such as the 7-pentoxyresorufin O-dealkylase activity of the CYP2B10 CAR target gene, may be measured (Pellinen et al. Hepatology 23: 515-23, 1996). Examples of CAR target genes include CYP2B10, a CYP2B6 transgene, CYP3A11, and a CYP3A4 transgene; examples of CAR responsive promoters include the CYP2B10, CYP2B6, CYP3A11, and CYP3A4 promoters and promoters operably-linked to DNA binding sites for CAR/RXR heterodimers. Alternatively, an increase in CAR receptor activity can be assayed by determining an increase in liver mass relative to total body mass, an increase in release of a liver enzyme such as alanine aminotransferase into the serum, or an increase in DNA synthesis in the liver, using the assays described herein. CAR-mediated induction may be measured in response to a number of xenobiotic compounds, including TCPOBOP.

By “mutation” is meant an alteration in the nucleic acid sequence such that the amino acid sequence encoded by the nucleic acid sequence has at least one amino acid alteration as compared to the naturally-occurring sequence.

The mutation may, without limitation, be an insertion, deletion, frameshift mutation, or missense mutation. Alternatively, the mutation may alter the sequence of a CAR receptor promoter, transcriptional regulatory sequence, or translational regulatory sequence such that a smaller amount of CAR mRNA or protein is expressed. Preferably, the mutation results in at least a 25, 35, 50, 70, 80, 90, 95, 99, or 100% reduction in the activity of the encoded CAR receptor compared to the activity of a naturally-occurring CAR receptor. In another preferred embodiment, the level of induction of a CAR target gene in response to a xenobiotic administered to a mouse having a mutation in a CAR receptor is less that 10, 5, or 2-fold times the corresponding level of induction in a CAR null mouse that does not express CAR mRNA or protein.

By “transgenic” is meant any cell or organism which includes a DNA sequence which is inserted by artifice into a cell and becomes part of the genome of the organism which develops from that cell. As used herein, the transgenic organism is generally a transgenic non-human mammal, preferably, a rodent such as a mouse.

By “substantially eliminates CAR receptor activity” is meant reduces the CAR receptor activity by 25, 35, 50, 70, 80, 90, 95, 99, or 100% compared to the activity of a naturally-occurring CAR receptor. In another preferred embodiment, the level of residual CAR receptor activity is no greater than 10, 5, or 2 times the corresponding level of CAR receptor activity in a CAR null mouse that does not express CAR mRNA or protein.

By “a substantially active murine CAR receptor” is meant having at least 30, 60, 80, 90, 95, or 100% of the CAR receptor activity of the naturally-occurring murine CAR receptor encoded by GenBank Accession No. 2267575 in a normal murine host (Choi et al., J. Biol. Chem. 272: 23565-23571, 1997) (SEQ ID NO: 1).

By “a human CAR receptor” is meant a protein that has an amino acid sequence at least 75, 80, 90, 95, 99, or 100% identical to the amino acid sequence of the naturally-occurring human CAR receptor, encoded by GenBank Accession No. 458541 (Baes et al., Mol. Cell. Bio. 14: 1544-1551, 1994) (SEQ ID NO: 2), and that has at least 50, 75, 80, 90, 95, or 100% of the CAR receptor activity of a naturally-occurring human CAR receptor assayed under identical conditions. It is also contemplated that the expressed human CAR receptor may be a fragment having an amino acid sequence at least 75, 80, 90, 95, 99, or 100% identical to the corresponding region of a naturally-occurring human CAR receptor and having at least 60, 80, 90, 95, or 100% of the CAR receptor activity of a naturally-occurring human CAR receptor. In addition, a human CAR receptor is inhibited by clotrimazole, an inverse agonist of human, but not murine, CAR (Moore et al., J Biol Chem. 275: 15122-15127, 2000).

By “activation of a CAR receptor” is meant an increase in the rate of the CAR-mediated induction of a CAR target gene, or a transgene operably-linked to a CAR responsive promoter. Preferably, the increased induction of the CAR target gene or transgene in a mouse results in a 2, 5, 10, or 20-fold increased level of the encoded mRNA or protein, increased enzymatic activity of the CAR target gene, increased relative liver mass, increased release of a liver enzyme such as alanine aminotransferase into the serum, or increased DNA synthesis in the liver, as measured using the assays described herein. In another preferred embodiment, the increased induction is 2, 5, 10, or 20-fold greater in a mouse having wild-type CAR receptor activity than in a mouse having a mutation that reduces CAR receptor activity.

In one preferred embodiment, the candidate activator of a CAR receptor and a CAR receptor inverse agonist are administered to a mouse having a mutation that reduces CAR receptor activity or a mouse expressing a human CAR receptor. The level of induction of a CAR target gene is measured in the presence and absence of the candidate activator to determine whether the candidate activator effects an increase in the level of induction of the CAR target gene. The administration of the CAR receptor inverse agonist may decrease the initial level of induction of the CAR target gene and thus facilitate the detection of a increase in the induction mediated by the candidate activator.

By “inhibit a CAR receptor” is meant decrease the rate of induction of a CAR target gene or transgene operably-linked to a promoter of a CAR target gene. Preferably, the decreased induction of the CAR target gene or transgene in a mouse results in a 2, 5, 10, or 20-fold decreased level of the encoded mRNA, protein, enzymatic activity, relative liver mass, release of a liver enzyme into the serum, or DNA synthesis in the liver, as determined using the assays described herein. In another preferred embodiment, the decrease in the level of induction is 2, 5, 10, or 20-fold greater in a mouse having wild-type CAR receptor activity than in a mouse having a mutation that reduces CAR receptor activity.

In one preferred embodiment, the candidate inhibitor of a CAR receptor and a CAR receptor agonist are administered to a mouse having a mutation that reduces CAR receptor activity or a mouse expressing a human CAR receptor.

The level of induction of a CAR target gene is measured in the presence and absence of the candidate inhibitor to determine whether the candidate inhibitor effects a decrease in the level of induction of the CAR target gene. The administration of the CAR receptor agonist may increase the initial level of induction of the CAR target gene and thus facilitate the detection of a decrease in the induction mediated by the candidate inhibitor.

By “having wild-type CAR receptor activity” is meant having a substantially identical activity to that of a naturally-occurring murine or human CAR receptor. By “substantially identical,” as used herein, is meant at least 80, 90, 95, 99, or 100% of the activity of a naturally-occurring CAR receptor.

The ability of a CAR receptor to induce a CAR target gene or a transgene operably-linked to a CAR responsive promoter may be routinely measured using assays for the encoded mRNA, protein, or enzymatic activity or assays for relative liver mass, a liver enzyme released into the serum, or DNA synthesis.

By “modulate the metabolism” is meant to increase or decrease the rate of a CYP-catalyzed reaction of a compound, such as the oxidation of the compound. For example, the rate of metabolism of the compound may be measured as the rate of formation of the oxidized product or the formation of a subsequent product generated from the oxidized intermediate. Alternatively, the rate of metabolism may be represented as the half-life or rate of disappearance of the initial compound or as the change in toxicity or activity of the initial compound or a metabolite generated in a CYP-dependent manner from the initial compound. For example, a second compound is said to modulate the metabolism of a first compound if the half-life, toxicity, or activity of the first compound is increased or decreased in the presence of the second compound. Preferably, the change in the half-life, toxicity, or activity of the first compound or a metabolite of the first compound is at least 25, 50, 100, 200, 500, or 1000% of the corresponding half-life, toxicity, or activity in the absence of the second compound. In another preferred embodiment, the change in the half-life, toxicity, or activity is at least 2, 5, 10, or 20-fold greater in a mouse having wild-type CAR receptor activity than in a mouse having a mutation that reduces CAR receptor activity. In various preferred embodiments, a second compound mediates a change of at least 2, 5, 10, or 20-fold in the magnitude of the half-life, activity, or toxicity of a first compound or a metabolite of the first compound, as measured in any of the assays described herein.

The half-life may be measured by determining the amount of the compound present in samples taken from the mouse at various time points; the amount of the compound may be quantified using standard methods such as high-performance liquid chromatography, mass spectrometry, western blot analysis using compound specific antibodies, or any other appropriate method.

In preferred embodiments, a reaction required for the toxicity or activity of the first compound or a metabolite of the first compound (such as the reaction of an activated metabolite with DNA, RNA, or protein) is at least 25, 50, 100, 200, 500, or 1000% of the corresponding rate in the absence of the second compound. The toxicity of the first compound or a metabolite of the first compound may also be measured by determining the relative liver mass, amount of a liver enzyme released into the serum, or rate of DNA synthesis in the liver of a mouse. It is also contemplated that the rate of a reaction catalyzed by another enzyme involved in xenobiotic metabolism that is downstream of a CAR receptor may also be increased or decreased. In one preferred embodiment, the second compound modulates the metabolism of the first compound by activating or inhibiting a CAR receptor.

By “activity of a compound” is meant a biological effect mediated by a compound. Examples of possible activities of compounds include binding to other molecules, modulation of a binding interaction between molecules, modulation of the rate of catalysis of an enzyme, induction of physiological or behavioral changes, or any other therapeutically relevant activity of a compound.

By “physiological effect” is meant a toxic effect, an activity, or the modulation of the expression of a CAR target gene mediated by a compound, as described above. For compounds that are metabolized to form a metabolite that has a different level of toxicity or activity as the initial compound, the physiological effect of the compound may also be measured by determining the half-life of the compound.

By “promoter” is meant a minimal sequence sufficient to direct transcription of an operably-linked gene. The promoter may also be operably-linked to 5′ regulatory sequences that modulate the transcription of the gene.

The present invention provides a number of advantages. For example, the methods of the present invention may be used to facilitate the identification of analogs of a compound that have reduced or undetectable ability to activate a CAR receptor, and thus are expected to have fewer side-effects or a longer half-life in vivo. In addition, because murine and human CAR receptors have somewhat different substrate specificities, the use of transgenic mice expressing a human CAR receptor in the methods of the present invention may more accurately predict the modulation of CAR receptor toxicity or half-life of a compound when administered to humans. Moreover, the present assays may be easily and rapidly performed.

Other features and advantages of the invention will be apparent from the following detailed description and from the claims.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1A is a schematic illustration of the targeted disruption of the mouse CAR gene. Boxes represent exons. Exons 2 and 3 (hatched boxes) contain the DNA binding domain. Homologous recombination resulted in replacement of Exons 1 and 2 with the β-gal and neo resistance genes. Restriction enzyme sites for Apa I (A), Hind III (H3), Not I (N), Sal I (S), and Xba I (X) are indicated;

FIG. 1B is a picture of a gel showing genotype analysis by Southern blotting. Genomic DNA from tail samples was digested with Hind III and hybridized with the 3′ probes. The 10 Kb and 15 Kb bands were generated from wild-type and mutant alleles, respectively;

FIG. 1C is a picture of a gel showing Northern blot analysis. The murine CAR cDNA was used as a probe to determine the level of CAR mRNA expressed in liver of wild-type and CAR+/− or −/− animals;

FIG. 2A is a picture of a representative Northern blot of xenobiotic activation of the CYP2B10 gene by CAR in a liver sample. Mice (8-10 weeks old, 3 mice per treatment) were treated with corn oil (CO) for 6 or 24 hours, PB for 24 hours, or TCPOBOP for 6 hours.

FIG. 2B is a series of photographs of in situ hybridization of a piece of small intestine from mice treated with PB or TCPOBOP for 3 days. The in situ hybridization was performed with an [³⁵S]-labeled antisense CYP2B10 riboprobe. The number of grains per cell are not significantly different in the CAR−/− animals, with or without xenobiotic treatment. The number of grains per cell is approximately two-fold higher in the untreated wild-type mice than in the CAR receptor knockout mice, and the number of grains per cell in the xenobiotic-treated wild-type mice is approximately two-fold higher than in the untreated wild-type mice.

FIG. 3A is a bar graph showing the liver enlargement and hepatocyte proliferation by PB or TCPOBOP. Mice (8-10 weeks old) were treated with PB or TCPOBOP for 3 days, and then both liver mass and body weight were measured. The data is presented as percentage of liver mass relative to total body weight.

FIG. 3B is a set of pictures of PB-treated, TCPOBOP-treated, or control mice that were treated with BrdU for two hours before their liver tissues were harvested. The representative microphotography illustrates the presence of BrdU-positive hepatocytes only in PB- or TCPOBOP-treated wild-type animals.

FIG. 4 is a bar graph showing the effect of PB or TCPOBOP on cocaine-mediated hepatotoxicity, measured as serum alanine aminotransferase (ALT) activity. Male mice were pretreated with PB or TCPOBOP for three days. Twenty-four hours after the last dose, one injection of cocaine was given to the animals. Blood was drawn 20 hours after cocaine treatment for determination of serum ALT activity.

FIGS. 5A and 5B are pictures of representative Northern blots of xenobiotic activation of the murine CYP3A11 gene by murine CAR in a liver sample. Mice (8-10 weeks old, 3 mice per treatment) were treated with corn oil (CO) for 6 or 24 hours, PB for 24 hours, or TCPOBOP for 6 hours.

FIG. 6 is a schematic illustration of the transgene construct used to generate mice expressing human CAR. This transgenic construct contains the liver specific, albumin promoter operably linked to the cDNA sequence for human CAR. To enhance the expression and stability of human CAR transcripts, a region from an abundantly expressed gene, rabbit-globin, and the polyadenylation (poly A) sequence from bovine growth hormone were also added to this construct.

FIGS. 7A and 7C are pictures of representative Southern blots of genomic DNA from mice generated using the human CAR transgene construct. The lanes containing DNA that bound to the human CAR probe are labeled with as asterisk.

FIG. 7B is a representative gel showing the PCR amplification of genomic DNA from mice generated using the human CAR transgene construct. The production of a PCR product using primers specific for human CAR confirmed the results of the Southern blot analysis in FIGS. 7A and 7C.

Based on these analyses, nine of the mice were identified as transgenic mice containing DNA encoding human CAR.

FIG. 8 is a Northern blot illustrating expression of human CAR mRNA transcripts in one of the humanized CAR mice lines (line 6210). As expected, human CAR mRNA was specifically expressed in the liver.

FIG. 9 is a bar graph illustrating the induction of a CAR reporter gene in HepG2 cells transiently transfected with the human CAR transgenic construct illustrated in FIG. 6. These results indicate that this transgenic construct encodes functional human CAR which can activate the expression of a reporter gene operably liked to a CAR responsive promoter. These transfected cells may be used to screen candidate compounds to determine whether they activate or inhibit human CAR.

### DETAILED DESCRIPTION OF THE INVENTION

The present screening methods and systems stem from the discovery that mice lacking the nuclear hormone receptor Constitutive Androstane Receptor (CAR or NR114) gene have decreased metabolism of the classic CYP substrate zoxazolamine and are not able to activate expression of the CYP2B10 gene or produce liver hypertrophic or hyperplastic responses upon treatment with either phenobarbital or the more potent inducer TCPOBOP. In contrast, strong activation and toxicity were seen in wild-type mice. In addition, cocaine treatment in the presence of either inducer resulted in acute hepatotoxicity in wild-type mice, but no detectable toxicity in CAR−/− “knockout” mice. Accordingly, the present invention provides screening methods for comparing the activation of CAR target genes, toxicity, and half-life of compounds after administration to mice with reduced or no CAR receptor activity versus administration to wild-type mice. These methods allow the identification of compounds that activate CAR receptors and are potentially toxic to mammals (e. g., humans), as well as compounds that inhibit CAR receptors and reduce the toxicity or CYP-mediated metabolism of a pharmaceutically active compound administered to a mammal.

CAR Receptor Knockout Mice

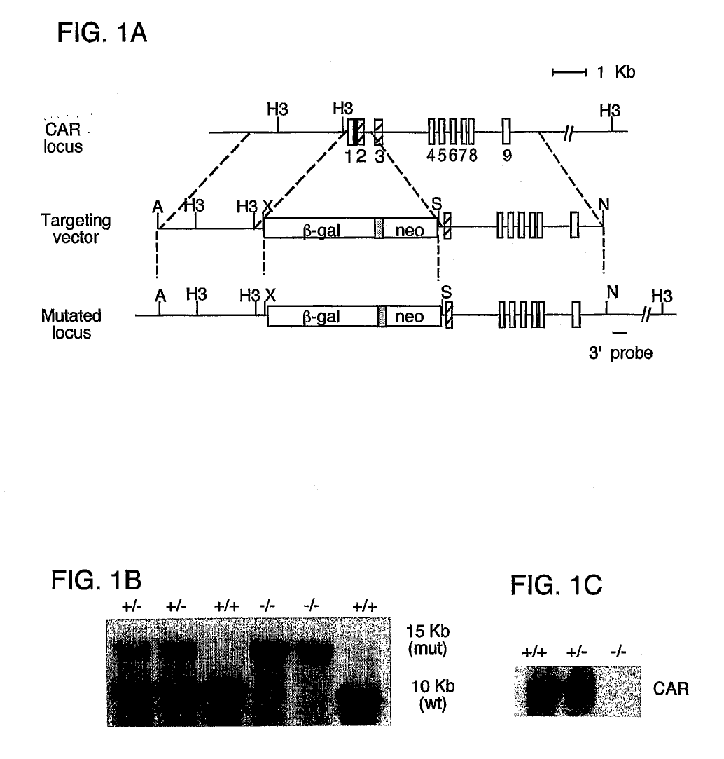

To assess the functional role of CAR, we generated two independent mouse lines in which a promoter proximal segment of the CAR gene, including a portion of the DNA binding domain, was replaced by the coding region for p-galactosidase (FIG. 1A). As expected, these (β-galactosidase “knockin” animals were unable to express CAR mRNA (FIG. 1C). This loss of CAR expression did not result in any overt phenotype; homozygous CAR−/− animals were born at expected Mendelian frequency, and both male and female−/− animals were fertile.

CAR has previously been reported to be expressed predominantly in the liver. To define the pattern of CAR expression in more detail, p-galactosidase expression was examined in CAR+/− heterozygotes. As expected, the β-galactosidase marker was expressed in liver, and expression was highest near the portal vessels. p-galactosidase expression was also observed in the epithelial cells of the small intestine.

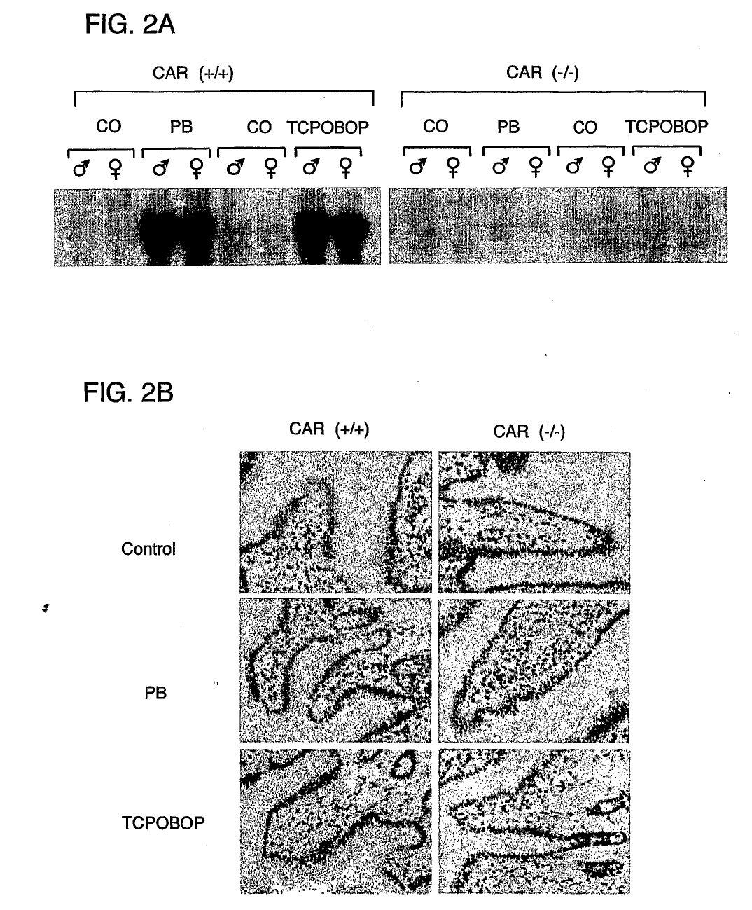

To test the role of CAR in the response to PB-like inducers, the effect of treating wild-type and CAR−/− animals with either PB or TCPOBOP was examined. The robust induction of expression of CYP2B10 mRNA in response to either of these two compounds in wild-type male or female animals was completely absent in the knockout animals (FIG. 2A). Similar results were obtained with both independent CAR−/− lines. This requirement for CAR was also demonstrated in the small intestine, using in situ hybridization. As indicated in FIG. 2B, either PB or TCPOBOP also induced CYP2B10 expression in this tissue in wild-type, but not CAR−/− animals. The confinement of specific hybridization to the epithelial cells was consistent with previous results and also with the pattern of CAR expression described above.

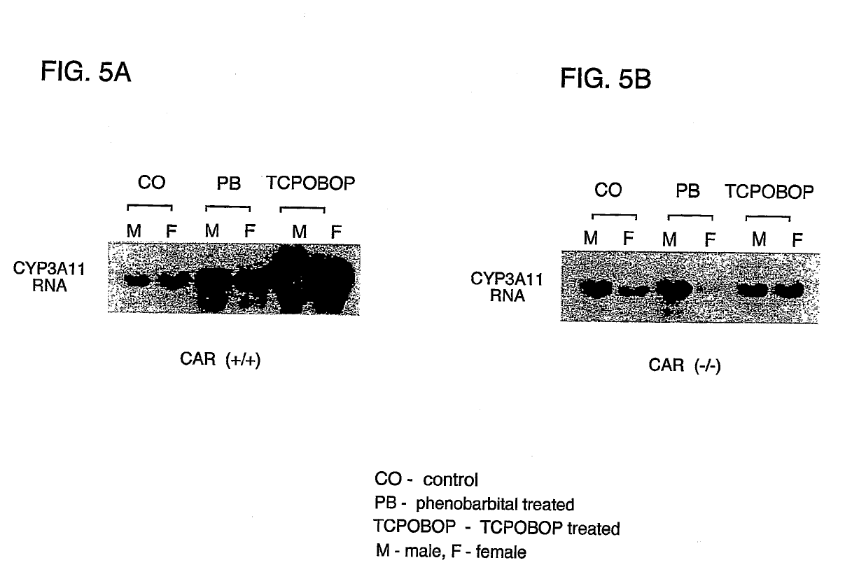

Similar to the induction of CYP2B10, a substantial induction of murine CYP3A11 mRNA in response to PB or TCPOBOP was observed in wild-type mice (FIG. 5A). In contrast, a negligible level of induction of CYP311 was detected in control CAR−/− mice (FIG. 5B).

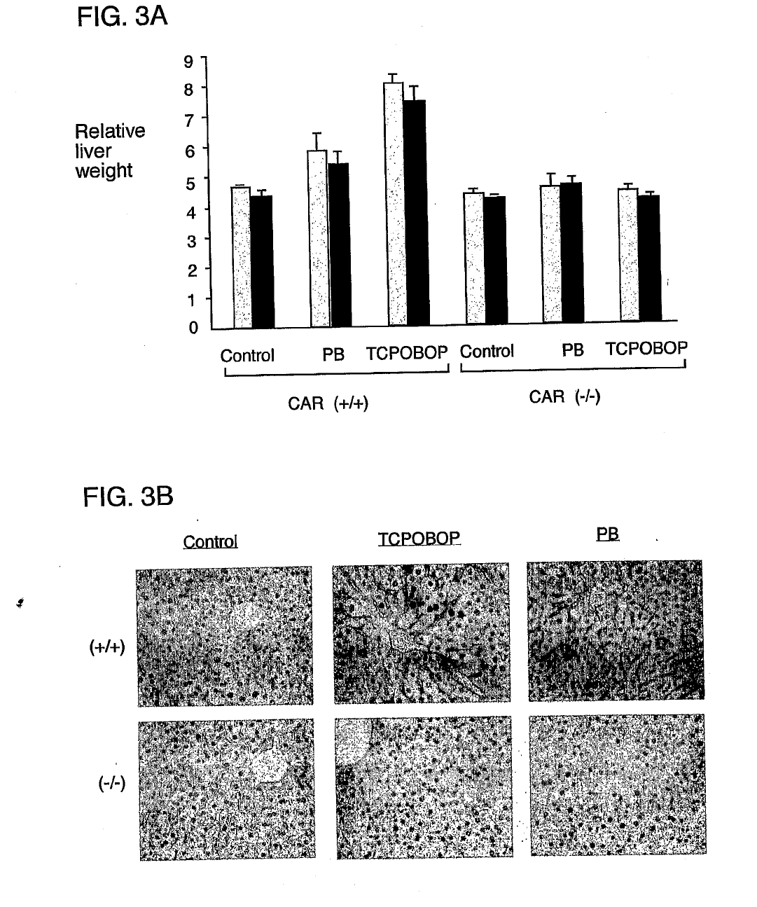

Acute treatments with PB-like inducers, particularly TCPOBOP, cause an up to 2-fold increase in liver mass relative to total body mass. This hepatomegaly is thought to be a reflection of both cellular hypertrophy and mitogenesis. The CAR−/− mice showed no evidence of the increase in liver mass observed in the wild-type mice after 3 days of treatment with either PB or TCPOBOP (FIG. 3A). The xenobiotic induction of DNA synthesis revealed by increased incorporation of BrdU in the wild-type animals was also completely absent in the CAR−/− animals (FIG. 3B).

These results demonstrate that CAR is essential for these responses to PB-like inducers. This conclusion was confirmed and extended by examination of the effect of the loss of CAR expression on metabolism of two xenobiotics.

The first is the classic substrate zoxazolamine. Many studies have demonstrated that increased CYP enzyme activity results in increased metabolic inactivation of this muscle relaxant, which is reflected in decreased duration of zoxazolamine-induced paralysis. As demonstrated in Tables 1 and 2, pretreatment of wild-type animals with either PB or TCPOBOP significantly decreased the duration of paralysis, as expected. The duration of paralysis was substantially longer in untreated CAR−/− mice than in wild-type mice, and, consistent with the results described above, the paralysis was not affected by pretreatment with either PB or TCPOBOP. For example, wild-type control female mice were paralyzed more than 12 hours, while wild-type xenobiotic-pretreated female mice were not paralyzed. Among the CAR−/− females, two animals from each group of control, PB-treated, or TCPOBOP-treated animals died; the survivors were paralyzed for more than 12 hours.

TABLE 1
Increased duration of zoxazolamine-induced paralysis in male CAR
(−/−) mice due to decreased metabolism of zoxazolamine

| | CAR (+/+) | | CAR (−/−) |
|---|---|---|---|
| Control | 2 | hours | >5 hours |
| PB-treated | <20 | minutes | >5 hours |
| TCPOBOP-treated | <20 | minutes | >5 hours |

TABLE 2
Increased duration of zoxazolamine-induced paralysis in female CAR
(−/−) mice due to decreased metabolism of zoxazolamine

| | CAR (+/+) | CAR (−/−) |
|---|---|---|
| Control | >12 hours | died |
| PB-treated | not paralyzed | died |
| TCPOBOP-treated | not paralyzed | died |

In these experiments, mice were pretreated for three days with PB or TCPOBOP, after which time they were given a single intraperitoneal injection of zoxazolamine (300 mg/kg). Paralysis time was recorded as the time when the mice were able to right themselves repeatedly.

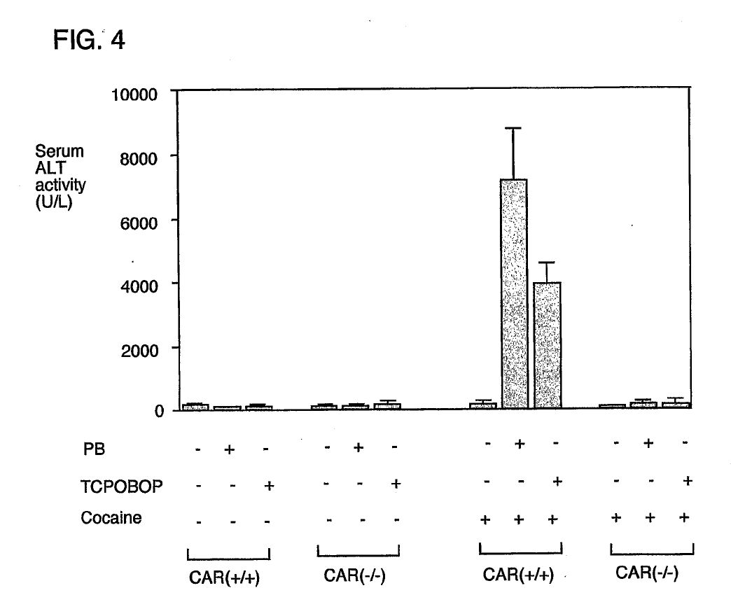

Treatment with PB-like inducers also sensitizes animals to hepatotoxic effects of a number of compounds, including cocaine. As shown in FIG. 4, treatment with either PB or TCPOBOP resulted in a significant increase in serum levels of the liver enzyme alanine aminotransferase (ALT) as an acute response to cocaine administration. This evidence of liver damage was not observed in CAR−/− animals.

These results clearly demonstrated that CAR was required for response to PB-like inducers of xenobiotic metabolism, and thus CAR functioned as a xenobiotic receptor in vivo to mediate the response to PB-like inducers. CAR can therefore be added to the previously described peroxisome proliferator activated receptor a and the aryl hydrocarbon receptor as a primary determinant of the response of phase I metabolic enzymes to foreign compounds. CAR is joined in this by its closest relative within the receptor superfamily, PXR/SXR, which has recently been shown to mediate response to a distinct group of xenobiotics. Although both DNA binding specificity and xenobiotic responses of CAR and PXR/SXR have been reported to overlap to some extent, no evidence for any compensatory effect of the latter was observed in the CAR knockout animals. Thus, it is now apparent that specific xenobiotics can induce specific metabolic responses by activating distinct receptors.

This mechanism may account for a large number of clinically significant drug-drug interactions in which the presence of one compound, such as phenobarbital, results in increased metabolism of another drug or foreign compound. Differences in the levels of activation of xenobiotic receptors among individuals based on differences in exposure to specific xenobiotics may also explain the significant inter-individual variability of the levels of particular cytochromes. Consistent with the very low basal levels of mouse CYP2B10, most humans have low or undetectable levels of CYP2B6, a human target of CAR activation. However, this enzyme is present at up to 100-fold higher levels in a subset of individuals. The results described here suggest that this variability could be the basis for the relatively rare but clinically significant hepatotoxicity observed in a subset of individuals exposed to high levels of cocaine. More generally, variations in CAR activity in response to the wide range of PB-like inducers may have significant impact on the metabolism of a wide range of pharmacologic agents and other foreign compounds. The CAR mice described herein facilitate the identification of compounds able to activate CAR in vivo, as well as enable identification of additional, specific downstream target genes that mediate its effects.

The experiments described above were carried out as follows:

Targeting vector construction. To construct the targeting vector for the CAR locus, an Xba I-Eag I fragment containing the nuclear localized β-galactosidase gene from vector pPD 46.21 was subcloned into the Xba I and EcoR I sites of the pGKneo plasmid. DNA from AB1 ES cells was used to amplify CAR genomic fragments for both 5′ and 3′ arms. For the 5′ arm, a 3 kb CAR promoter fragment was cloned into the Apa I and Xba I sites. For the 3′ arm, a 5 kb fragment spanning exons 3 to 9 was cloned into the Sal I and Not I sites. The primers for the 5′ arm were 5′-gcgcgcgggccctggcatacattaacacaaacacatacatat-3′ (SEQ ID NO: 3) and 5′-gcgcgctctagaaggacccagactctggacccagggcaaaga-3′ (SEQ ID NO: 4). The primers for the 3′ arm were 5′-gcgcgcgtcgacaggtgaagtgcttctccccaacagaaacaa-3′ (SEQ ID NO: 5) and 5′-gcgcgcgcggccgctgtcctgggagcagcctctgcagccgct-3′ (SEQ ID NO: 6).

Generation of CAR receptor knockout mice. AB1 ES cells (10⁷) were electroporated with 25 μg targeting construct in 0.9 ml PBS using a Bio-Rad Gene Pulser (500 μF, 230 V). The cells were then plated on one or two 10-cm plates containing a monolayer of irradiated STO feeder cells. Twenty-four hours later, they were subjected to G418 selection (350, μg/ml, Gibco) for 9 days. Resistant clones were analyzed by Southern blotting after Hind III digestion, using the 3′ probe indicated in FIG. 1A (FIG. 1B). The primers for the 3′ probe were 5′-ggacaacctcagcccacagtgatgc-3′ (SEQ ID NO: 7) and 5′-tcctttggttaccacctgactctgc-3′ (SEQ ID NO: 8). Two positive clones were expanded and injected into C57BL/6 blastocysts. Male chimeras were back crossed to C57BL/6 females. Heterozygotes were determined by Southern blotting and intercrossed to generate homozygotes.

Animal treatment. At least three mice between 8-10 weeks old were used for each treatment. Mice were pretreated by intraperitoneal injection with corn oil, PB (100 mg/kg, Sigma), or TCBOPOP (3 mg/kg) for the indicated time. For the three day PB treatment, mice were injected intraperitoneally three times with PB, one injection per day.

Zoxazolamine paralysis test. Mice pretreated with corn oil, PB, or TCPOBOP were given a single intraperitoneal injection of zoxazolamine (300 mg/kg, Sigma) 24 hours after the last dose of PB. Mice were placed on their backs, and the paralysis time was defined as the time required for the animal to regain sufficient consciousness to right itself repeatedly (Liang et al., Proc. Natl. Acad. Sci. USA, 93: 1671-6, 1996).

Cocaine treatment and ALT assay. Male mice pretreated with corn oil, PB, or TCPOBOP were injected intraperitoneally with cocaine HCl (30 mg/kg) 24 hours after the last dose of PB. The mice were anaesthetized 24 hours after cocaine treatment. Blood was drawn from the eye for determination of serum alanine aminotransferase (ALT) activity.

RNA analysis. 20 μg of total RNA from individual mouse livers was subjected to Northern blot analysis (FIG. 1C). A mouse CAR cDNA probe was used to reveal the absence of CAR transcripts in the CAR null mice. Probes for CYP2B10 were prepared by RT-PCR with mouse liver total RNA using Superscript One-step RT-PCR System (Life Technologies). PCR primers were 5′-ccgcctctagaagtcaacattggttagac-3′ (SEQ ID NO: 9) and 5′-ccgccggatcccacactaagcctcataat-3′ (SEQ ID NO: 10). For in situ hybridization, small intestine tissue was cross sectioned at 7 μM thickness. Slides were subjected to in situ hybridization with a [³⁵S]-labeled CYP2B10 antisense probe. To prepare the probe, the CYP2B10 RT-PCR product was subcloned into the Xba I and Bam HI sites of Bluescript® SK(−) phagemid (Stratagene). The plasmid was linearized with Xba 1. T7 RNA polymerase was used to synthesize [³⁵S]-labeled antisense probes.

Determination of proliferation of hepatocytes following PB or TCPOBOP treatment. Mice pretreated with corn oil, PB or TCPOBOP received a single intraperitoneal dose of BrdU/FdU (2 ml/100 g, Amersham). Mice were sacrificed 2 hours after BrdU administration. BrdU incorporation was determined using a mouse anti-BrdU monoclonal antibody (DAKO Corporation) and Vectastain ABC Kit (Vector Laboratories Inc.) (FIG. 3B), using standard procedures.

Generation of Mice Expressing a Human CAR Receptor. It is known that the ligand binding domains of human and mouse CAR genes differ somewhat in amino acid sequence, and that these two proteins respond differently to some activators. In particular, TCPOBOP is an agonist ligand for the murine CAR receptor, but not the human CAR receptor (Tzameli et al., supra). Similarly, clotrimazole is an inverse agonist for the human, but not the murine, CAR receptor (Moore et al., supra). As a result, CAR−/− knockout mice expressing a functional human CAR receptor also provide useful models for drug screening since their response to drugs or other xenobiotic compounds should be based on the human rather than the murine CAR receptor. Such “humanized” CAR mice allow for the identification of compounds, such as those in clinical development, with potentially undesirable effects in humans, which may not be evident in mice.

A humanized CAR mouse lacking the murine CAR but expressing the human CAR receptor may be generated by any of several standard methods (see, for example, Ausubel et al. (Chapter 9), supra). For example, a conventional transgenic animal expressing the human CAR gene from a promoter active in appropriate tissues, such as the liver, may be generated. Examples of such promoters include those directing expression of albumin (Xie et al., Nature 406: 435-439, 2000), transthyretin (Ye et al., Mol Cell Biol. 19: 8570-8580, 1999), or CAR itself. This human CAR transgene may then be introduced into a homozygous CAR−/− mouse by conventional breeding (Pierson et al., Mol. Endocrinol. 14: 1075-1085, 2000; Slee et al., Proc Natl Acad Sci USA. 96: 8040-8045, 1999). In another possible method, the human CAR transgene may be injected into fertilized oocytes from homozygous CAR−/− mice, directly generating the desired transgenic mice. In a third method, embryonic stem cells may be generated from a homozygous CAR−/− animal (Ausubel et al. (Chapter 9), supra). Conventional homologous recombination techniques may then be used to replace the inactivated murine CAR gene with a functional human CAR receptor gene (Fiering et al., Methods Enzymol. 306: 42-66, 1999). Since the CAR−/− animals contain the neo gene which confers resistance to G418, another appropriate gene such as hygromycin may be used in the human CAR replacement construct to allow the selection of cells in which the human CAR gene has replaced the inactivated murine CAR gene. In still another possible method, a functional human CAR receptor gene may be introduced into a homozygous CAR−/− mouse using gene therapy and a promoter active in appropriate tissues, such as the promoters described above, and contained in an adenoviral, adeno-associated viral, retroviral, lentiviral, herpes viral, nonviral, or any other suitable vector (see, for example, Sarkar et al., Hum Gene Ther. 11: 881-894, 2000; Goddard et al., Gene Ther. 4: 1231-1236, 1997).

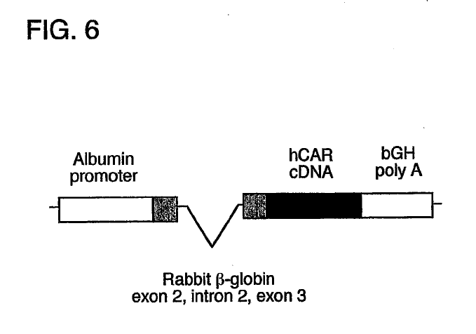

Transgene Construct for Generation of Mice Expressing a Human CAR Receptor. In one of the possible methods for the generation of mice expressing a human CAR receptor, the transgene construct illustrated in FIG. 6 was used. This transgenic construct contains the coding sequence for a human CAR receptor operably linked to the liver specific, albumin promoter. Additionally, a region from an abundantly expressed gene, rabbit β-globin, was added between the promoter and the CAR receptor coding sequence to enhance the expression of the human CAR receptor. The polyadenylation (poly A) sequence from bovine growth hormone was also added downstream of the human CAR receptor coding sequence. The bovine growth hormone is a strong poly A sequence that ensures termination of transcription and stabilization of the mRNA transcripts. Furthermore, the use of a heterologous poly A sequence eliminates the requirement to isolate the genomic sequence which corresponds to the endogenous poly A sequence of human CAR.

The transgenic human CAR construct was generated from several previously described plasmids. To generate the starting vector, a BamHI-EcoRI 640 base pair fragment consisting of part of exon 2, intron 2, and exon 3 of the rabbit β-globin gene [nucleotides˜551-1190 from Entrez accession number V00878, from the previously described vector pKCR (Nikaido et al., Nature 311: 631-635, 1984)] was inserted into the corresponding sites in the pBluescript plasmid (Stratagene) to generate vector KCR-KS. A 350 base-pair blunt-ended XbaI-XhoI fragment from the bovine growth hormone polyadenlyation signal (“bGHpA,” containing nucleotides 1671-1867 of Entrez accession number AF335419) was removed from the PGKNeo plasmid (Mortensen et al., Mol. Cell Biol. 12 (5): 2391-5, 1992) and inserted into the EcoRV site of the KCR-KS vector to generate the KbpA vector.

To facilitate cloning of the human CAR cDNA insert into the KbpA vector, we introduced an annealed oligonucleotide consisting of restriction sites AvrII, StuI, BglII, EcoRV, and EcoRI downstream of the original EcoRI site and upstream of the bGHpA site to generate the KbpAlb vector. A XbaI-NotI (gap-filled), human CAR cDNA (“hCAR,” 1.2 kilobases, GenBank Accession No. 458541) was then subcloned into the AvrII-EcoRV sites of the KbpAlb vector to obtain the KbpAlb-hCAR construct. A 2.5 kb HindIII-BamHI fragment consisting of KCR, hCAR cDNA, and bGHpA was then ligated into the corresponding sites of a modified bluescript 3′sk vector to yield vector KbpAlb-hCAR3′sk. The modified bluescript 3′sk vector that was used for this step contained unique eight base-pair clusters such as AscI, SwaI, and Pad cleavage sites that replaced the ClaI-Acc65I region in the 3′ end of the original bluescript SK vector. Lastly, a 2.3 kb fragment containing the albumin promoter digested with NotI and BamHI (obtained from Ronald Evans, Xie et al., supra) was cloned into the NotI and BamHI sites of the KbpAlb-hCAR3′sk vector to obtain the Alb-hCAR transgenic construct. This plasmid contains an ampicillin resistance gene and the ColEl origin of replication.

Generation and Characterization of Humanized CAR Mice Using the Alb-hCAR Transgene Construct. For the generation of humanized CAR mice, fertilized one-celled embryos collected from C57BL/6 donor females mated with C57BL/6 males were collected in M2 media and microinjected with the linearized Alb-hCAR transgenic construct described above. The resulting mice were tested as described below to determine whether they expressed human CAR. If desired, mice expressing human CAR but lacking mouse CAR can be generated by mating humanized CAR mice with the CAR−/− knockout mice described above. Alternatively, ES cells or embryos from humanized CAR mice can be genetically modified as described above to disrupt the endogenous mouse CAR gene.

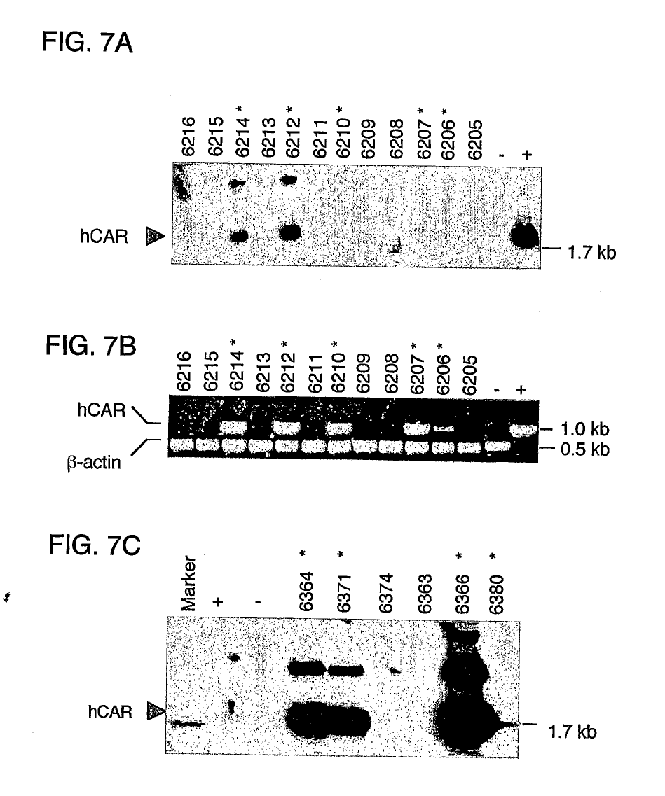

The integration and retention of the transgenic construct in the mice was confirmed by standard Southern blotting analysis. For this analysis, genomic DNA was digested with BamHI and Asp718 and then probed with a 1 kb fragment containing the BamHI-EcoRI restriction enzyme-digested sequence encoding the human CAR ligand binding domain (LBD). The human LBD region was used as the probe because the LBD is the region that is the least homologous to murine CAR. The expected band of about 1.7 kb indicates that a mouse is a transgenic mouse containing the human CAR coding sequence (FIGS. 7A and 7C, * denotes lanes identified as containing DNA from a transgenic mouse). Nine transgenic founder mice were identified based on this analysis.

To further confirm that these transgenic mice contained DNA encoding human CAR, PCR analysis was performed. Primers hCAR-hinge5′ (5′-CCGGAATTCAGGAAAGACATGATACTGTCGGCAGAAGCC-3′, SEQ ID NO: 15) and hCAR3′ (5′-cgcggatccGGCCGCTGCAGGCGCAGAACTGGTAGGTATGG-3′, SEQ ID NO: 16) were used to specifically amplify the human CAR cDNA sequence and generate an PCR product of 1000 base pairs (FIG. 7B). As a positive control, primers SCBF (5′-GAT GTG CTC CAG GCT AAA GTT-3′, SEQ ID NO: 17) and SCBR (5′-AGA AAC GGA ATG TTG TGG AGT-3′, SEQ ID NO: 18) were used to amplify endogenous mouse β-actin to produce a PCR product of 600 base pairs. This analysis confirmed that the nine founder mice contain human CAR DNA.

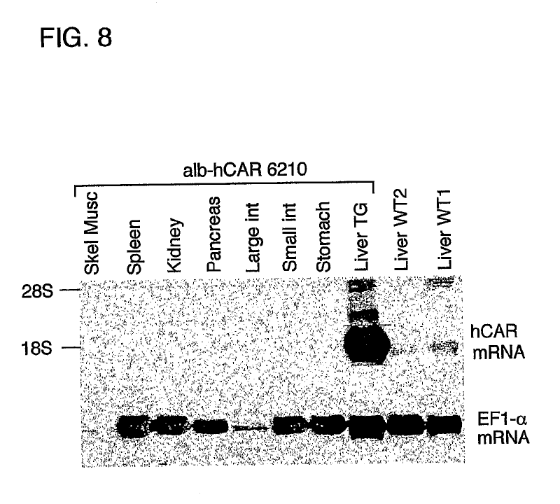

Four of the nine mice lines were also tested by Northern blot analysis to determine if they expressed human CAR mRNA transcripts of the expected size (˜1.5 kb). For this analysis, cellular mRNA was probed with the same probe to the LBD region that was used for the Southern blot analysis. One of the mice lines expressed a human CAR mRNA transcript of the expected size (FIG. 8). As expected, the human CAR mRNA, which was under the control of the liver specific albumin promoter, was specifically expressed in the liver of this transgenic line. The other three lines appeared to express mRNA transcripts that were larger than the expected size. These larger mRNA transcripts may indicate rearrangement of the transgene or inappropriately-spliced structures. All of the four lines were analyzed for human CAR expression in at least the following organs: the liver, spleen, small intestine, and pancreas.

Assays for CAR Receptor Activity. The homozygous CAR−/− animals described herein are useful for drug metabolism assays since they allow the identification of drugs or other xenobiotic compounds that induce expression of CYP2B10, CYP3A11, or other CAR target genes in wild-type, but not CAR−/− animals. The detection of CYP2B10 or CYP3A11 induction may be performed by any of several assays, including assays of CYP2B10 or CYP3A11 protein levels (for example, by Western blot analysis), mRNA levels (for example, by Northern blot analysis), or enzymatic activity (for example, my measuring 7-pentoxyresorufin O-dealkylase enzymatic activity as described, for example, in Pellinen et al. (Hepatology 23: 515-23, 1996). Alternatively, an increase in liver mass relative to total body mass or an increase in DNA synthesis in the liver may be measured as described herein. Similar assays for other CAR target genes may also be used.

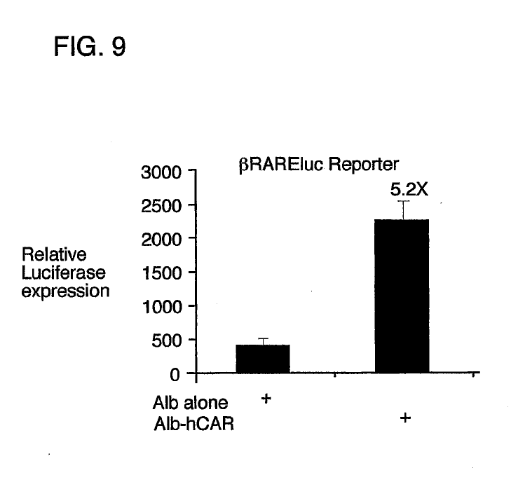

Moreover, as alternatives to assays involving endogenous murine CAR target genes, assays may be conducted to measure appropriate reporter transgenes inserted by any standard technique (for example, those techniques described above) into wild-type mice, CAR−/− mice, humanized CAR mice, mice lacking the gene encoding the receptor related to CAR (known as SXR, PXR or by other names (Kliewer et. al., Cell 92: 73-82, 1998; Blumberg et al., Genes Dev. 12: 3195-3205, 1998)), or any other appropriate strain. These reporter transgenes consist of a CAR responsive promoter operably-linked to an easily measured reporter gene. Examples of appropriate promoters include native CYP promoters such as the CYP2B10 promoter containing the previously described phenobarbital response element (Honkakoski et al., supra), the CYP2B6 promoter, the CYP3A11 promoter, the CYP3A4 promoter, or synthetic promoter constructs in which DNA binding sites for CAR/RXR heterodimers are linked to functional basal promoters (Tzameli, et al., supra). Examples of appropriate reporter genes include, without limitation, human growth hormone, secreted alkaline phosphatase, luciferase, green fluorescent protein, chloramphenicol acetyl transferase, CYP2B6, CYP3A11, and any other reporter gene (see, for example, Ausubel et al. (Chapter 9), supra). The assays for CAR target genes involve standard procedures (see, for example, Ausubel et al. (Chapter 9), supra) and may be based on appropriate samples from the mice, such as liver or serum samples. Alternatively, hepatocytes or other appropriate cell types may be harvested from the animals and propagated, Compounds may be administered to these cells to determine whether the compounds effect a change in expression of CAR target genes or reporter transgenes. Candidate compounds may also be tested for their ability to activate or inhibit human CAR in cell based assays using cells that have been transiently or stably transfected with a transgenic construct encoding human CAR. For example, the Alb-hCAR transgenic construct illustrated in FIG. 6 which encodes human CAR under the control of the albumin promoter was used to transiently transfect a human hepatoma derived HepG2 cell line. The HepG2 cell lines contains a plasmid with a previously described reporter construct, denoted betaRAREluciferase, which contains a luciferase reporter gene under the control of a CAR responsive promoter (Forman et al., Nature 395 (6702): 612-5, 1998). As illustrated in FIG. 9, transfection of the cells with the Alb-hCAR transgene construct resulted in a 5.2-fold greater level of luciferase reporter gene expression than transfection of the cells with the corresponding control construct which lacks the human CAR coding sequence. This result indicates that this transgenic construct encodes functional human CAR which can activate the expression of a reporter gene operably liked to a CAR responsive promoter. The cells transfected with the Alb-hCAR construct can be used to identify candidate compounds that increase the expression of the luciferase reporter gene as activators of human CAR and to identify candidate compounds that decrease the expression of the luciferase reporter gene as inhibitors of human CAR.

Other Embodiments. From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A transgenic mouse expressing a human CAR receptor.
